(19) United States
(12) Patent Application Publication
(10) Pub. No.: US 2016/0007933 A1
(43) Pub. Date: Jan. 14, 2016

(54) SYSTEM AND METHOD FOR PROVIDING A SMART ACTIVITY SCORE USING EARPHONES WITH BIOMETRIC SENSORS

(71) Applicant: JAYBIRD LLC, Salt Lake City, UT (US)

(72) Inventors: STEPHEN DUDDY, Moama (AU); BEN WISBEY, Canberra (AU); DAVID SHEPERD, Canberra (AU); HAGEN DIESTERBECK, Little Bay (NZ); JUDD ARMSTRONG, Parrearra (AU)

(73) Assignee: JAYBIRD LLC, Salt Lake City, UT (US)

(21) Appl. No.: 14/863,404

(22) Filed: Sep. 23, 2015

Related U.S. Application Data
(63) Continuation-in-part of application No. 14/137,734, filed on Dec. 20, 2013.
Application No. 14/137,734 is a continuation-in-part of application No. 14/062,815, filed on Oct. 24, 2013.
Application No. 14/062,815 is a continuation-in-part of application No. 14/830,549, filed on Aug. 19, 2015.

(51) Int. Cl. A61B 5/00; A61B 5/0205; A61B 5/11

(52) U.S. Cl. CPC A61B 5/7264; A61B 5/6817; A61B 5/1118; A61B 5/4866; A61B 5/486; A61B 5/6898; A61B 5/0002; A61B 5/742; A61B 5/7475; A61B 5/0205

(57) ABSTRACT

A system for providing a smart activity score includes an earphone with a biometric sensor used for providing a smart activity score to a user. The system includes a movement monitoring module that monitors a movement to determine a metabolic loading associated with the movement during a score period. The system also includes a period activity score module that creates and updates a period activity score based on the metabolic loading and the movement. The period activity score is created and updated for the score period. In addition, the system includes a smart activity score module that creates and updates a smart activity score by aggregating a set of period activity scores.

### CROSS-REFERENCE TO RELATED APPLICATIONS

This application is a continuation-in-part of and claims the benefit of U.S. patent application Ser. No. 14/137,734, filed Dec. 20, 2013 titled “System and Method for Providing a Smart Activity Score,” which is a continuation-in-part of and claims the benefit of U.S. patent application Ser. No. 14/062,815, filed Oct. 24, 2013, titled “Wristband with Removable Activity Monitoring Device.” This application is also a continuation-in-part of and claims the benefit of U.S. patent application Ser. No. 14/830,549, filed Aug. 19, 2015, titled “Earphones with Biometric Sensors.” The contents of the Ser. No. 14/137,734 application, the Ser. No. 14/062,815 application, and the Ser. No. 14/830,549 application are each incorporated herein by reference in their entireties.

### TECHNICAL FIELD

The present disclosure relates generally to fitness monitoring devices, and more particularly to systems and methods for providing a smart activity score.

### DESCRIPTION OF THE RELATED ART

Previous generation movement monitoring and fitness tracking devices generally enabled only a tracking of activity that accounts for estimated total calories burned. Currently available fitness tracking devices now add functionality that use universal metabolic equivalent tasks to track activity and performance. Issues with currently available fitness tracking devices, however, include that they do not track user activities at a granular level, and do not tightly couple metabolic equivalents to user characteristics. Moreover, currently available solutions do not account in a precise manner for the health and performance benefits of sustained activity. The lack of precision and personalized functionality is due in part to the manner of data acquisition, as well as the rudimentary tracking methods and analysis employed.

### BRIEF SUMMARY OF THE DISCLOSURE

In view of the above drawbacks, there exists a long-felt need for fitness monitoring devices that track user activities at a granular level and that are tightly coupled to user characteristics. Further, there is a need for fitness monitoring devices that provide increased resolution into the performance benefits of sustained activity.

Embodiments of the present disclosure provide systems and methods for providing a smart activity score. Some particular embodiments of the present disclosure provide systems and methods for providing a smart activity score using earphones configured with biometric sensors (e.g. heartrate sensor, motion sensor, etc.) in communication with a computing device.

According to an embodiment of the technology disclosed herein, biometric earphones used in accordance with the disclosed technology include a battery; a circuit board electrically coupled to the battery; a first processor electrically coupled to the circuit board; a pair of earphones including speakers; a controller; and a cable electrically coupling the earphones to the controller. In one embodiment, one of the earphones includes an optical heartrate sensor electrically coupled to the first processor, and protruding from a side of the earphone proximal to an interior side of a user's ear when the earphone is worn; and a motion sensor electrically coupled to the first processor, where the first processor is configured to process electronic input signals from the motion sensor and the optical heartrate sensor. In various embodiments, the first processor is configured to calculate a heart rate variability value based on the signals received from the optical heartrate sensor.

In embodiments, the biometric earphones further include a second processor electrically coupled to the circuit board and configured to process electronic input signals carrying audio data. In alternative embodiments, the first processor is also configured to process electronic input signals carrying audio data.

In embodiments, the earphones include a wireless transmitter configured to transmit heart rate and motion data stored in a memory of the biometric earphones to a computing device configured to process the received biometric data and provide a smart activity score to a user. In a particular implementation, the wireless transmitter is a BLUETOOTH transmitter.

In embodiments, the computing device that receives biometric data from the disclosed earphones includes a display; one or more processors; and one or more non-transitory computer-readable mediums operatively coupled to at least one of the one or more processors and having instructions stored thereon that, when executed by at least one of the one or more processors, cause: at least one of the one or more processors to process the biometric data received from the activity monitoring device; and the display to display an activity display based on the processed biometric data.

In embodiments, the computing device includes a movement monitoring module that monitors movement to determine a metabolic loading associated with the movement. In some embodiments, the movement monitoring module can indirectly monitor movement by monitoring the biometric data received from the disclosed earphones in real-time or near real-time. The movement monitoring module monitors the movement during a score period. The computing device further includes a period activity score module that creates and updates a period activity score based on the metabolic loading and the movement. The period activity score is created and updated for the score period. In one embodiment, the score period is ten seconds. The computing device also includes a smart activity score module that creates and updates a smart activity score by aggregating a set of period activity scores.

In one embodiment, the metabolic loading is determined from a set of metabolic loadings, each metabolic loading being determined according to user information from a user. The smart activity score is associated with a measuring period. In such embodiments, the smart activity score module calculates an average smart activity score from a set of past smart activity score. Each past smart activity score is associated with a past measuring period. The user information includes a user lifestyle selected from a set of reference lifestyles.

In embodiments, the computing device used to provide a smart activity score further includes a user lifestyle module. The user lifestyle module associates each reference lifestyle with a lower threshold score and an upper threshold score. The lower threshold score and the upper threshold score associated with each reference lifestyle define a range or scores. No two ranges of scores overlap. The user lifestyle module changes the user lifestyle to the reference lifestyle associated with the lower threshold score not greater than the average smart activity score and the upper threshold score not less than the average smart activity score.

In one embodiment, the computing device includes a period activity score multiplier module that applies a period activity score multiplier to the period activity score to create an adjusted period activity score. In such an embodiment, the smart activity score includes an aggregation of adjusted period activity scores. The period activity score multiplier, in one embodiment, is directly proportional to the number of continuous score periods over which a user activity type of the movement and a user activity intensity of the movement is maintained. In a further embodiment, the period activity score multiplier is directly proportional to the smart activity score for the current measuring period. Although modules disclosed herein have been described as being embodied in the computing device, one or more of the modules may be embodied in the sensors of the disclosed earphones. In particular, in various embodiments at least one of the movement monitoring module, the period activity score module, and the smart activity score module is embodied in a sensor (e.g. motion sensor, optical heartrate sensor, etc.) of the disclosed earphones, the earphones being configured to be attached to the body of a user (e.g. worn in a user's ears).

The disclosure, in one embodiment, involves a method for providing a smart activity score. The method includes monitoring a movement to determine a metabolic loading associated with the movement. The movement is monitored during a score period. The score period, in one embodiment, is ten seconds. The method also includes creating and updating a period activity score based on the metabolic loading and the movement. The period activity score is created and updated for the score period. The method further includes creating and updating a smart activity score by aggregating a set of period activity scores.

In one embodiment, the metabolic loading is determined from a set of metabolic loadings and each metabolic loading is determined according to user information from a user. The user information includes a user lifestyle selected from a set of reference lifestyles. Determining the set of metabolic loadings is based on the user lifestyle. The method, in one embodiment, includes associating each reference lifestyle with a lower threshold score and an upper threshold score. The lower threshold score and the upper threshold score associated with each reference lifestyle define a range of scores. No two ranges of scores overlap. In one embodiment, the method includes calculating an average smart activity score from a set of past smart activity scores. Each past smart activity score is associated with a past measuring period. In one embodiment, the method includes changing the user lifestyle to the reference lifestyle associated with the lower threshold score not greater than the average smart activity score and the upper threshold score not less than the average smart activity score.

In one embodiment, the method includes comparing the smart activity score to a past smart activity score. The smart activity score is associated with a measuring period. The past smart activity score is associated with a past measuring period. The method may include receiving a second smart activity score from a second user. In one embodiment, the method includes comparing the smart activity score to the second smart activity score.

The method for providing the smart activity score, in one embodiment, includes applying a score period multiplier to the period activity score to create an adjusted period activity score. The smart activity score, in such an embodiment, includes an aggregation of adjusted period activity scores. In one embodiment, the score period multiplier is directly proportional to the number of continuous score periods over which a user activity type of the movement and a user activity intensity of the movement is maintained. The score period multiplier, in a further embodiment, is directly proportional to the smart activity score for the current measuring period. Although operations disclosed herein have been described as being accomplished using the computing device, one or more operations may be accomplished using one or more sensors embodied in the disclosed earphones. In particular, in various embodiments, at least one of the operations of monitoring the movement, creating and updating the period activity score, and creating and updating the smart activity score is accomplished using a sensor (e.g. motion sensor, optical heartrate sensor, etc.) of the disclosed earphones, the earphones being configured to be attached to the body of a user (e.g. worn in a user's ears).

One embodiment includes a system for providing a smart activity score. The system includes a processor and at least one computer program residing on the processor. The computer program is stored on a non-transitory computer readable medium having computer executable program code embodied thereon. The computer executable program code is configured to monitor a movement to determine a metabolic loading associated with the movement during a score period. The computer executable program code is further configured to create and update a period activity score based on the metabolic loading and the movement during the score period. The computer executable program code is configured to create and update a smart activity score by aggregating a set of period activity scores.

Other features and aspects of the disclosed method and system will become apparent from the following detailed description, taken in conjunction with the accompanying drawings, which illustrate, by way of example, the features in accordance with embodiments of the disclosure. The summary is not intended to limit the scope of the claimed disclosure, which is defined solely by the claims attached hereto.

### BRIEF DESCRIPTION OF THE DRAWINGS

The present disclosure, in accordance with one or more various embodiments, is described in detail with reference to the following Figures. The Figures are provided for purposes of illustration only and merely depict typical or example embodiments of the disclosure.

FIG. 1 illustrates an example communications environment in which embodiments of the disclosed technology may be implemented.

FIG. 2A illustrates a perspective view of exemplary earphones that may be used to implement the technology disclosed herein.

FIG. 2B illustrates an example architecture for circuitry of the earphones of FIG. 2A.

FIG. 3A illustrates a perspective view of a particular embodiment of an earphone, including an optical heartrate sensor, in accordance with the disclosed technology.

FIG. 3B illustrates a side perspective view of placement of the optical heartrate sensor of the earphones of FIG. 3A when they are worn by a user.

FIG. 3C illustrates a frontal perspective view of placement of the optical heartrate sensor of the earphones of FIG. 3A when they are worn by a user.

FIG. 3D illustrates a perspective view of an over-the-ear configuration of dual-fit earphones in accordance with the disclosed technology.

FIG. 3E illustrates a perspective view of an over-the-ear configuration of the dual-fit earphones of FIG. 3D.

FIG. 3F illustrates a perspective view of an under-the-ear configuration of the dual-fit earphones of FIG. 3D.

FIG. 4A is a block diagram illustrating an example computing device that may be used to implement embodiments of the disclosed technology.

FIG. 4B illustrates modules of an example activity monitoring application that may be used to implement embodiments of the disclosed technology.

FIG. 4C illustrates exemplary processing modules embodied in the disclosed computing device in accordance with embodiments of the disclosed technology.

FIG. 4D illustrates further exemplary processing modules embodied in the disclosed computing device in accordance with embodiments of the disclosed technology.

FIG. 5 is an operational flow diagram illustrating a method of prompting a user to adjust the placement of earphones in the user's ear to ensure accurate biometric data collection by the earphones' biometric sensors.

FIG. 6 illustrates an activity display that may be associated with an activity display module of the activity monitoring application of FIG. 4B.

FIG. 7A is an operational flow diagram illustrating an example of a method for creating and updating a smart activity score.

FIG. 7B is an example of a metabolic loading table.

FIG. 7C is an example of an activity intensity library.

FIG. 8A is an operational flow diagram illustrating an example of a method for creating and updating a smart activity score including basing the smart activity score on a user lifestyle and changing the user lifestyle.

FIG. 8B is an operational flow diagram illustrating an example of a method for creating and updating a smart activity score including comparing the smart activity score to other smart activity scores.

FIG. 9 illustrates a sleep display that may be associated with a sleep display module of the activity monitoring application of FIG. 4B.

FIG. 10 illustrates an activity recommendation and fatigue level display that may be associated with an activity recommendation and fatigue level display module of the activity monitoring application of FIG. 4B.

FIG. 11 illustrates a biological data and intensity recommendation display that may be associated with a biological data and intensity recommendation display module of the activity monitoring application of FIG. 4B.

FIG. 12 illustrates an example computing module that may be used to implement various features of the technology disclosed herein.

### DETAILED DESCRIPTION

Embodiments of the present disclosure are directed toward systems and methods for providing a smart activity score. The disclosure is directed toward various embodiments of such systems and methods. In one such embodiment, the systems and methods implemented using an activity monitoring device that provides a smart activity score. According to some embodiments of the disclosure, the activity monitoring device may be a pair of earphones with biometric sensors, the earphones configured to be situated within a user's ears. In addition to wirelessly receiving high-fidelity audio data for playback, the disclosed earphones may collect the user's biometric data such as heartrate data and movement data, and wirelessly transmit the biometric data to a computing device for additional processing and user interaction via an activity tracking application installed on the computing device.

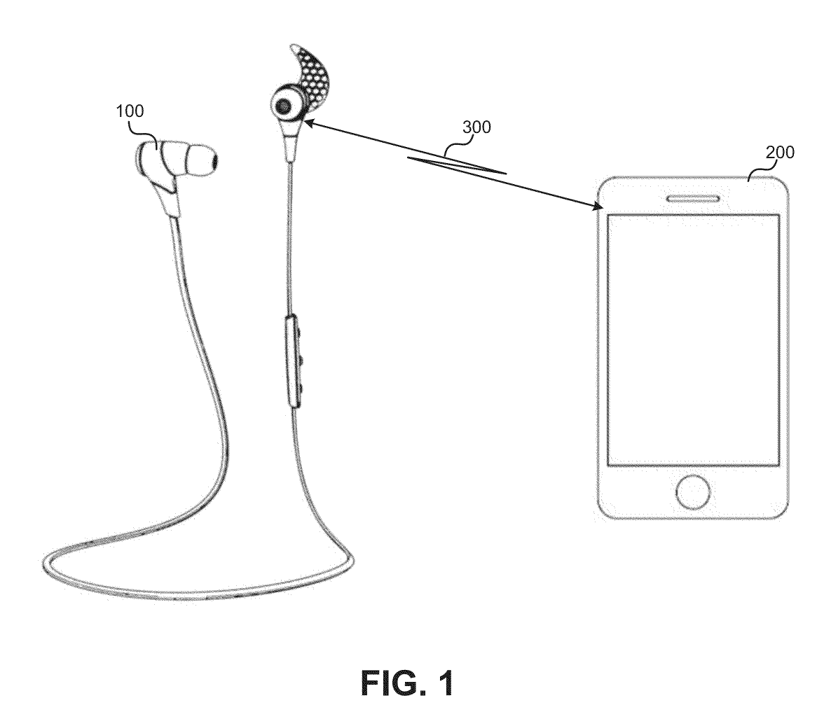

FIG. 1 illustrates an example communications environment in accordance with an embodiment of the technology disclosed herein. In this embodiment, earphones 100 communicate biometric and audio data with computing device 200 over a communication link 300. The biometric data is measured by one or more sensors (e.g., heart rate sensor, accelerometer, gyroscope) of earphones 100. Although a smartphone is illustrated, computing device 200 may comprise any computing device (smartphone, tablet, laptop, smartwatch, desktop, etc.) configured to transmit audio data to earphones 100, receive biometric data from earphones 100 (e.g., heartrate and motion data), and process the biometric data collected by earphones 100. In additional embodiments, computing device 200 itself may collect additional biometric information that is provided for display. For example, if computing device 200 is a smartphone it may use built in accelerometers, gyroscopes, and a GPS to collect additional biometric data.

Computing device 200 additionally includes a graphical user interface (GUI) to perform functions such as accepting user input and displaying processed biometric data to the user. The GUI may be provided by various operating systems known in the art, such as, for example, iOS, Android, Windows Mobile, Windows, Mac OS, Chrome OS, Linux, Unix, a gaming platform OS, etc. The biometric information displayed to the user can include, for example a summary of the user's activities, a summary of the user's fitness levels, activity recommendations for the day, the user's heart rate and heart rate variability (HRV), and other activity related information. User input that can be accepted on the GUI can include inputs for interacting with an activity tracking application further described below. Computing device 200 may take a variety of forms, such as a desktop or laptop computer, a smartphone, a tablet, a processor, a module, or the like. In addition, computing device 200 may be a processor or module embedded in a wearable sensor, a bracelet, a smartwatch, earphones, a piece of clothing, an accessory, and so on. For example, computing device 200 may be substantially similar to devices embedded in earphones 100. Computing device 200 may communicate with other devices over a communication medium similar to the communications medium used to implement communication link 300 in FIG. 1, with or without the use of a server. In one embodiment, computing device 200 includes processing modules. In various embodiments, processing modules may be used to perform various processes.

Communication link 300 may be implemented in a variety of forms. In embodiments, the communication link 300 is a wireless communication link based on one or more wireless communication protocols such as BLUETOOTH, Wi-Fi, 4G LTE, ZIGBEE, 602.11 protocols, Infrared (IR), Radio Frequency (RF), etc. In embodiments, communication link 300 may be implemented using an Internet connection, such as a local area network (“LAN”), a wide area network (“WAN”), a fiber optic network, internet over power lines, a hard-wired connection (e.g., a bus), and the like, or any other kind of network connection. Communication link 300 may be implemented using any combination of routers, cables, modems, switches, fiber optics, wires, radio, and the like. One of skill in the art will recognize other ways to implement communication medium 300.

Alternatively, the communications link 300 may be a wired link (e.g., using any one or a combination of an audio cable, a USB cable, etc.) Though not explicitly depicted in FIG. 1, a server may direct communications made over communications link 300. The server may be, for example, an Internet server, a router, a desktop or laptop computer, a smartphone, a tablet, a processor, a module, or the like. In one embodiment, a server directs communications between communications link 300 and computing device 200. For example, the server may update information stored on computing device 200, or the server may send information to computing device 200 in real time.

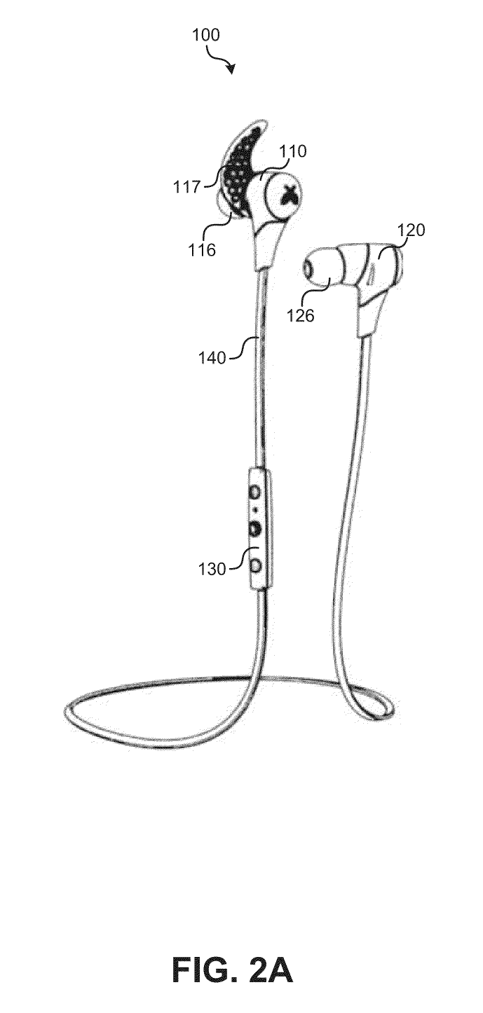

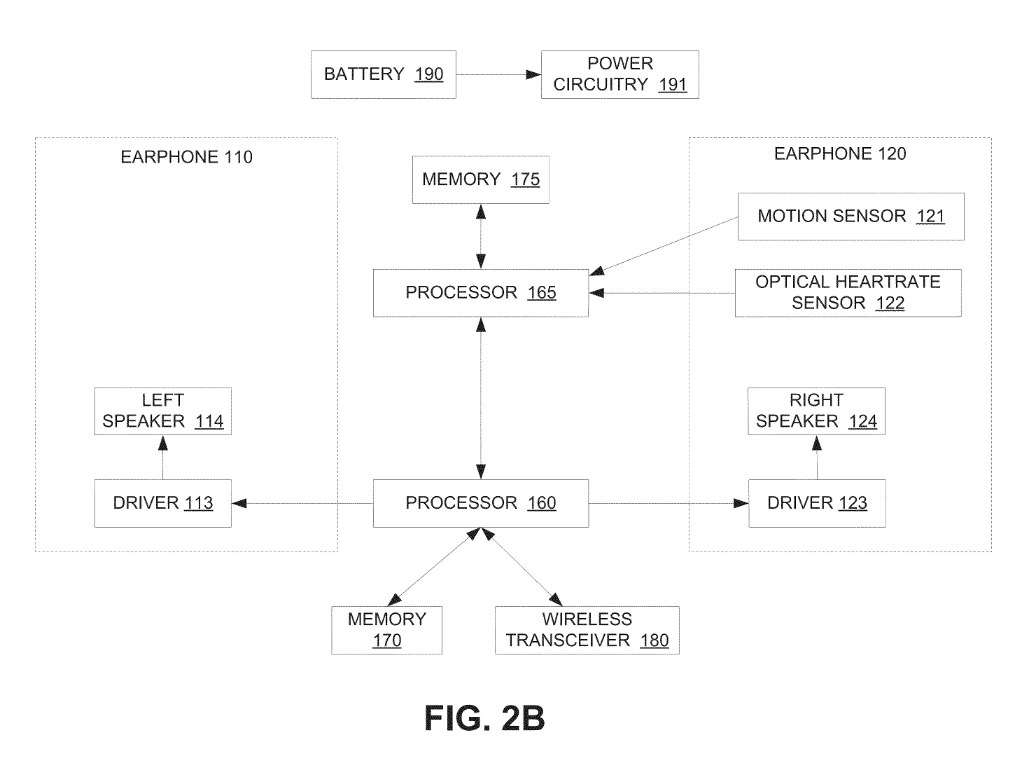

With specific reference now to earphones 100, FIG. 2A is a diagram illustrating a perspective view of exemplary earphones 100. FIG. 2A will be described in conjunction with FIG. 2B, which is a diagram illustrating an example architecture for circuitry of earphones 100. Earphones 100 comprise a left earphone 110 with tip 116, a right earphone 120 with tip 126, a controller 130 and a cable 140. Cable 140 electrically couples the right earphone 110 to the left earphone 120, and both earphones 110-120 to controller 130. Additionally, each earphone may optionally include a fin or ear cushion 117 that contacts folds in the outer ear anatomy to further secure the earphone to the wearer's ear.

In embodiments, earphones 100 may be constructed with different dimensions, including different diameters, widths, and thicknesses, in order to accommodate different human ear sizes and different preferences. In some embodiments of earphones 100, the housing of each earphone 110, 120 is rigid shell that surrounds electronic components. For example, the electronic components may include motion sensor 121, optical heartrate sensor 122, audio-electronic components such as drivers 113, 123 and speakers 114, 124, and other circuitry (e.g., processors 160, 165, and memories 170, 175). The rigid shell may be made with plastic, metal, rubber, or other materials known in the art. The housing may be cubic shaped, prism shaped, tubular shaped, cylindrical shaped, or otherwise shaped to house the electronic components.

The tips 116, 126 may be shaped to be rounded, parabolic, and/or semi-spherical, such that it comfortably and securely fits within a wearer's ear, with the distal end of the tip contacting an outer rim of the wearer's outer ear canal. In some embodiments, the tip may be removable such that it may be exchanged with alternate tips of varying dimensions, colors, or designs to accommodate a wearer's preference and/or fit more closely match the radial profile of the wearer's outer ear canal. The tip may be made with softer materials such as rubber, silicone, fabric, or other materials as would be appreciated by one of ordinary skill in the art.

In embodiments, controller 130 may provide various controls (e.g., buttons and switches) related to audio playback, such as, for example, volume adjustment, track skipping, audio track pausing, and the like. Additionally, controller 130 may include various controls related to biometric data gathering, such as, for example, controls for enabling or disabling heart rate and motion detection. In a particular embodiment, controller 130 may be a three button controller.

The circuitry of earphones 100 includes processors 160 and 165, memories 170 and 175, wireless transceiver 180, circuity for earphone 110 and earphone 120, and a battery 190. In this embodiment, earphone 120 includes a motion sensor 121 (e.g., an accelerometer or gyroscope), an optical heartrate sensor 122, and a right speaker 124 and corresponding driver 123. Earphone 110 includes a left speaker 114 and corresponding driver 113. In additional embodiments, earphone 110 may also include a motion sensor (e.g., an accelerometer or gyroscope), and/or an optical heartrate sensor.

A biometric processor 165 comprises logical circuits dedicated to receiving, processing and storing biometric information collected by the biometric sensors of the earphones. More particularly, as illustrated in FIG. 2B, processor 165 is electrically coupled to motion sensor 121 and optical heartrate sensor 122, and receives and processes electrical signals generated by these sensors. These processed electrical signals represent biometric information such as the earphone wearer's motion and heartrate. Processor 165 may store the processed signals as biometric data in memory 175, which may be subsequently made available to a computing device using wireless transceiver 180. In some embodiments, sufficient memory is provided to store biometric data for transmission to a computing device for further processing.

During operation, optical heartrate sensor 122 uses a photoplethysmogram (PPG) to optically obtain the user's heart rate. In one embodiment, optical heartrate sensor 122 includes a pulse oximeter that detects blood oxygenation level changes as changes in coloration at the surface of a user's skin. More particularly, in this embodiment, the optical heartrate sensor 122 illuminates the skin of the user's ear with a light-emitting diode (LED). The light penetrates through the epidermal layers of the skin to underlying blood vessels. A portion of the light is absorbed and a portion is reflected back. The light reflected back through the skin of the user's ear is then obtained with a receiver (e.g., a photodiode) and used to determine changes in the user's blood oxygen saturation (SpO2) and pulse rate, thereby permitting calculation of the user's heart rate using algorithms known in the art (e.g., using processor 165). In this embodiment, the optical sensor may be positioned on one of the earphones such that it is proximal to the interior side of a user's tragus when the earphones are worn.

In various embodiments, optical heartrate sensor 122 may also be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV.

In further embodiments, logic circuits of processor 165 may further detect, calculate, and store metrics such as the amount of physical activity, sleep, or rest over a period of time, or the amount of time without physical activity over a period of time. The logic circuits may use the HRV, the metrics, or some combination thereof to calculate a recovery score. In various embodiments, the recovery score may indicate the user's physical condition and aptitude for further physical activity for the current day. For example, the logic circuits may detect the amount of physical activity and the amount of sleep a user experienced over the last 48 hours, combine those metrics with the user's HRV, and calculate a recovery score. In various embodiments, the calculated recovery score may be based on any scale or range, such as, for example, a range between 1 and 10, a range between 1 and 100, or a range between 0% and 100%.

During audio playback, earphones 100 wirelessly receive audio data using wireless transceiver 180. The audio data is processed by logic circuits of audio processor 160 into electrical signals that are delivered to respective drivers 113 and 123 of left speaker 114 and right speaker 124 of earphones 110 and 120. The electrical signals are then converted to sound using the drivers. Any driver technologies known in the art or later developed may be used. For example, moving coil drivers, electrostatic drivers, electret drivers, orthodynamic drivers, and other transducer technologies may be used to generate playback sound.

The wireless transceiver 180 is configured to communicate biometric and audio data using available wireless communications standards. For example, in some embodiments, the wireless transceiver 180 may be a BLUETOOTH transmitter, a ZIGBEE transmitter, a Wi-Fi transmitter, a GPS transmitter, a cellular transmitter, or some combination thereof. Although FIG. 2B illustrates a single wireless transceiver 180 for both transmitting biometric data and receiving audio data, in an alternative embodiment, a transmitter dedicated to transmitting only biometric data to a computing device may be used. In this alternative embodiment, the transmitter may be a low energy transmitter such as a near field communications (NFC) transmitter or a BLUETOOTH low energy (LE) transmitter. In implementations of this particular embodiment, a separate wireless receiver may be provided for receiving high fidelity audio data from an audio source. In yet additional embodiments, a wired interface (e.g., micro-USB) may be used for communicating data stored in memories 165 and 175.

FIG. 2B also shows that the electrical components of headphones 100 are powered by a battery 190 coupled to power circuity 191. Any suitable battery or power supply technologies known in the art or later developed may be used. For example, a lithium-ion battery, aluminum-ion battery, piezo or vibration energy harvesters, photovoltaic cells, or other like devices can be used. In embodiments, battery 190 may be enclosed in earphone 110 or earphone 120. Alternatively, battery 102 may be enclosed in controller 130. In embodiments, the circuitry may be configured to enter a low-power or inactive mode when earphones 100 are not in use. For example, mechanisms such as, for example, an on/off switch, a BLUETOOTH transmission disabling button, or the like may be provided on controller 130 such that a user may manually control the on/off state of power-consuming components of earphones 100.

It should be noted that in various embodiments, processors 160 and 165, memories 170 and 175, wireless transceiver 180, and battery 190 may be enclosed in and distributed throughout any one or more of earphone 110, earphone 120, and controller 130. For example, in one particular embodiment, processor 165 and memory 175 may be enclosed in earphone 120 along with optical heartrate sensor 122 and motion sensor 121. In this particular embodiment, these four components are electrically coupled to the same printed circuit board (PCB) enclosed in earphone 120. It should also be noted that although audio processor 160 and biometric processor 165 are illustrated in this exemplary embodiment as separate processors, in an alternative embodiment the functions of the two processors may be integrated into a single processor.

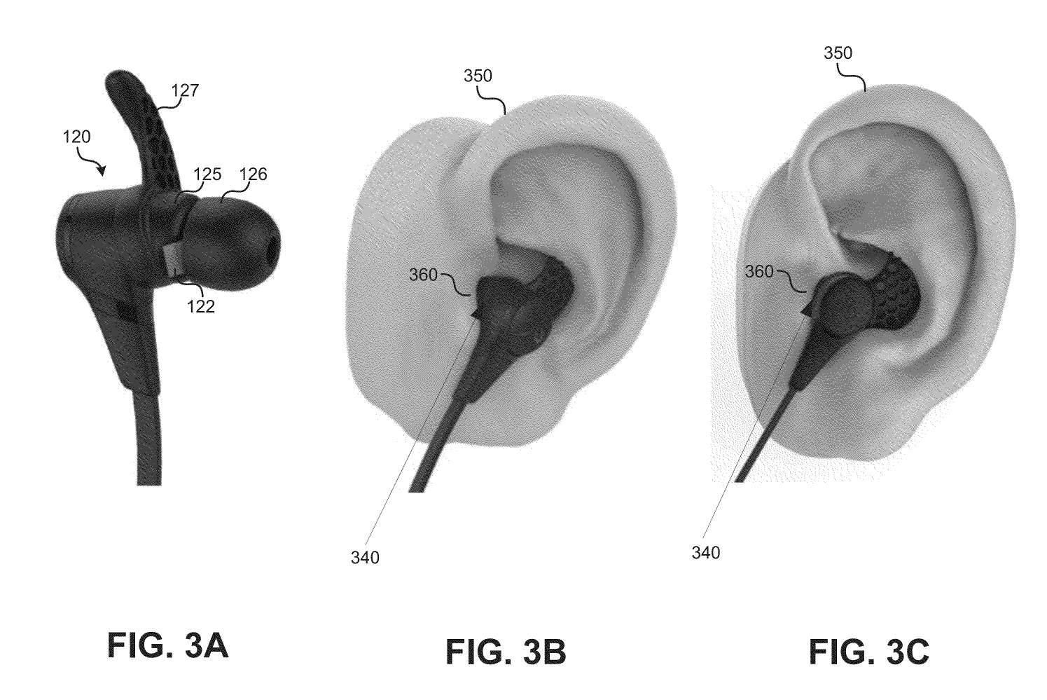

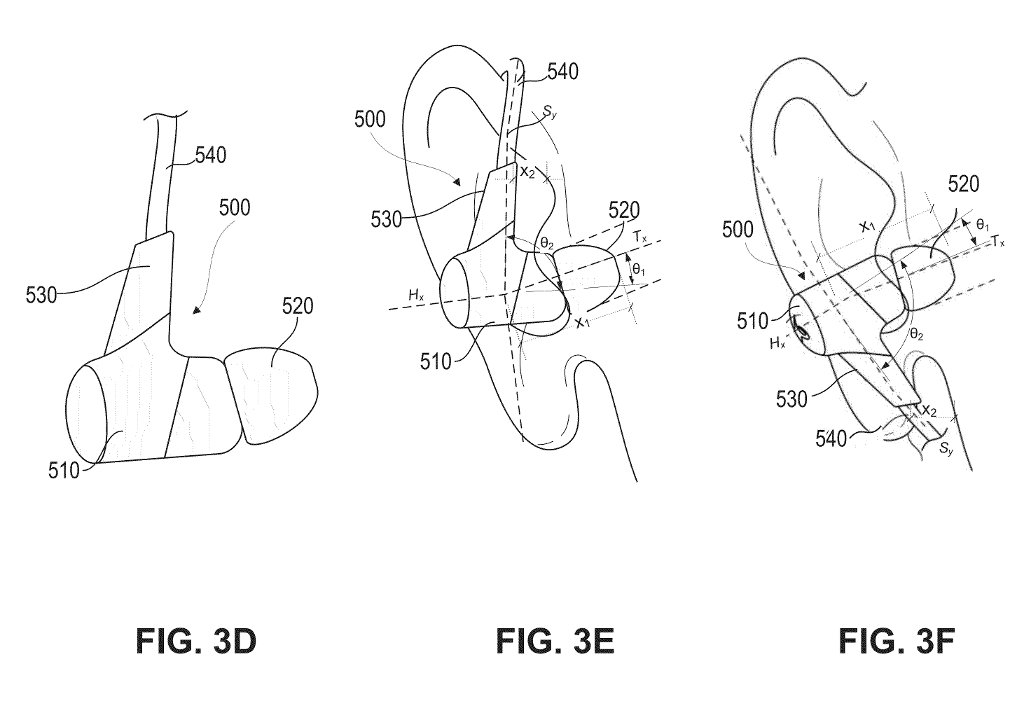

FIG. 3A illustrates a perspective view of one embodiment of an earphone 120, including an optical heartrate sensor 122, in accordance with the technology disclosed herein. FIG. 3A will be described in conjunction with FIGS. 3B-3C, which are perspective views illustrating placement of heartrate sensor 122 when earphone 120 is worn in a user's ear 350. As illustrated, earphone 120 includes a body 125, tip 126, ear cushion 127, and an optical heartrate sensor 122. Optical heartrate sensor 122 protrudes from a frontal side of body 125, proximal to tip 126 and where the earphone's nozzle (not shown) is present. FIGS. 3B-3C illustrate the optical sensor and ear interface 340 when earphone 120 is worn in a user's ear 350. When earphone 120 is worn, optical heartrate sensor 122 is proximal to the interior side of a user's tragus 360.

In this embodiment, optical heartrate sensor 122 illuminates the skin of the interior side of the ear's tragus 360 with a light-emitting diode (LED). The light penetrates through the epidermal layers of the skin to underlying blood vessels. A portion of the light is absorbed and a portion is reflected back. The light reflected back through the skin is then obtained with a receiver (e.g., a photodiode) of optical heartrate sensor 122 and used to determine changes in the user's blood flow, thereby permitting measurement of the user's heart rate and HRV.

In various embodiments, earphones 100 may be dual-fit earphones shaped to comfortably and securely be worn in either an over-the-ear configuration or an under-the-ear configuration. The secure fit provided by such embodiments keeps the optical heartrate sensor 122 in place on the interior side of the ear's tragus 360, thereby ensuring accurate and consistent measurements of a user's heartrate.

FIGS. 3D and 3E are cross-sectional views illustrating one such embodiment of dual-fit earphones 500 being worn in an over-the-ear configuration. FIG. 3F illustrates dual-fit earphones 500 in an under-the-ear configuration.

As illustrated, earphone 500 includes housing 510, tip 520, strain relief 530, and cord or cable 540. The proximal end of tip 520 mechanically couples to the distal end of housing 510. Similarly, the distal end of strain relief 530 mechanically couples to a side (e.g., the top side) of housing 510. Furthermore, the distal end of cord 540 is disposed within and secured by the proximal end of strain relief 530. The longitudinal axis of the housing, Hₓ, forms angle θ₁ with respect to the longitudinal axis of the tip, Tₓ. The longitudinal axis of the strain relief, S_{y}, aligns with the proximal end of strain relief 530 and forms angle θ₂ with respect to the axis Hₓ. In several embodiments, θ₁ is greater than 0 degrees (e.g., Tₓ extends in a non-straight angle from Hₓ, or in other words, the tip 520 is angled with respect to the housing 510). In some embodiments, θ₁ is selected to approximate the ear canal angle of the wearer. For example, θ₁ may range between 5 degrees and 15 degrees. Also in several embodiments, θ₂ is less than 90 degrees (e.g., S_{y}, extends in a non-orthogonal angle from Hₓ, or in other words, the strain relief 530 is angled with respect to a perpendicular orientation with housing 510). In some embodiments, θ₂ may be selected to direct the distal end of cord 540 closer to the wearer's ear. For example, θ₂ may range between 75 degrees and 85 degrees

As illustrated, x₁ represents the distance between the distal end of tip 520 and the intersection of strain relief longitudinal axis S_{y} and housing longitudinal axis Hₓ. One of skill in the art would appreciate that the dimension x₁ may be selected based on several parameters, including the desired fit to a wearer's ear based on the average human ear anatomical dimensions, the types and dimensions of electronic components (e.g., optical sensor, motion sensor, processor, memory, etc.) that must be disposed within the housing and the tip, and the specific placement of the optical sensor. In some examples, x₁ may be at least 18 mm. However, in other examples, x₁ may be smaller or greater based on the parameters discussed above.

Similarly, as illustrated, x₂ represents the distance between the proximal end of strain relief 530 and the surface wearer's ear. In the configuration illustrated, θ₂ may be selected to reduce x₂, as well as to direct the cord 540 towards the wearer's ear, such that cord 540 may rest in the crevice formed where the top of the wearer's ear meets the side of the wearer's head. In some embodiments, θ₂ may range between 75 degrees and 85 degrees. In some examples, strain relief 530 may be made of a flexible material such as rubber, silicone, or soft plastic such that it may be further bent towards the wearer's ear. Similarly, strain relief 530 may comprise a shape memory material such that it may be bent inward and retain the shape. In some examples, strain relief 530 may be shaped to curve inward towards the wearer's ear.

In some embodiments, the proximal end of tip 520 may flexibly couple to the distal end of housing 510, enabling a wearer to adjust θ₁ to most closely accommodate the fit of tip 520 into the wearer's ear canal (e.g., by closely matching the ear canal angle).

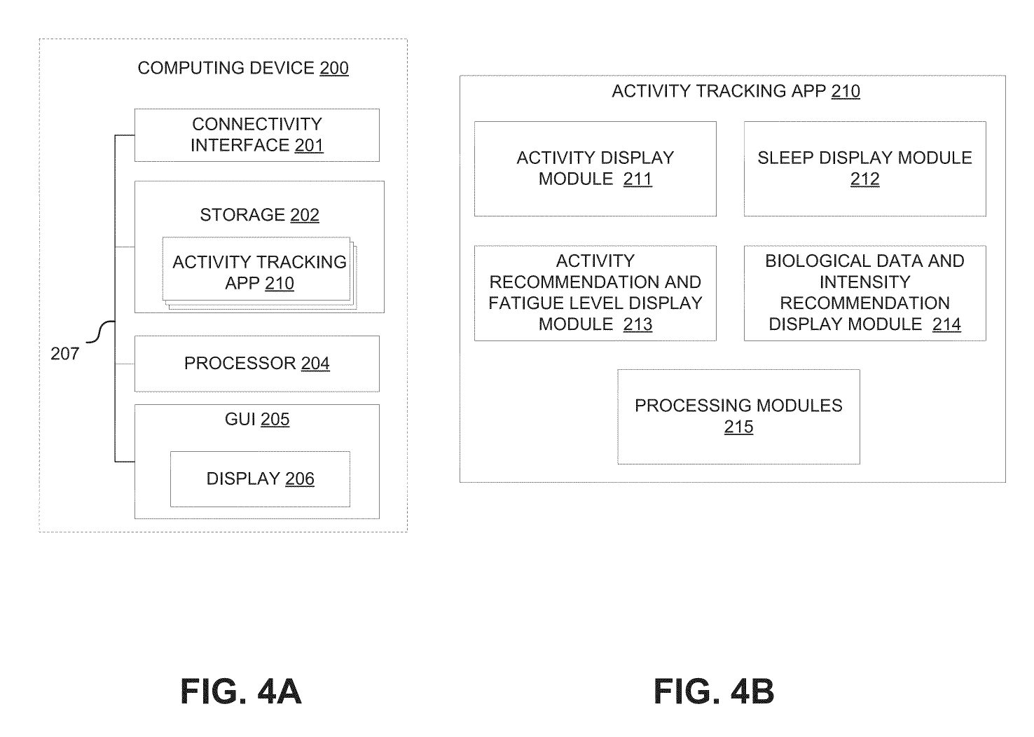

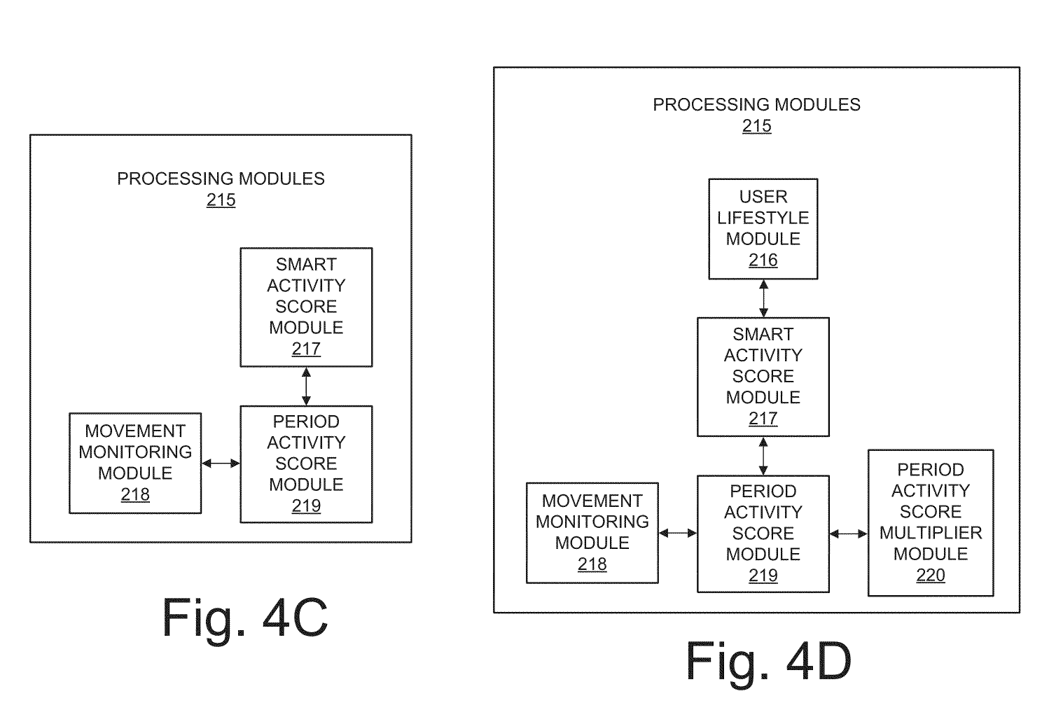

As one having skill in the art would appreciate from the above description, earphones 100 in various embodiments may gather biometric user data that may be used to track a user's activities and activity level. That data may then be made available to a computing device, which may provide a GUI for interacting with the data using a software activity tracking application installed on the computing device. FIG. 4A is a block diagram illustrating example components of one such computing device 200 including an installed activity tracking application 210.

As illustrated in this example, computing device 200 comprises a connectivity interface 201, storage 202 with activity tracking application 210, processor 204, a graphical user interface (GUI) 205 including display 206, and a bus 207 for transferring data between the various components of computing device 200.

Connectivity interface 201 connects computing device 200 to earphones 100 through a communication medium. The medium may comprise a wireless network system such as a BLUETOOTH system, a ZIGBEE system, an Infrared (IR) system, a Radio Frequency (RF) system, a cellular network, a satellite network, a wireless local area network, or the like. The medium may additionally comprise a wired component such as a USB system.

Storage 202 may comprise volatile memory (e.g. RAM), non-volatile memory (e.g. flash storage), or some combination thereof. In various embodiments, storage 202 may store biometric data collected by earphones 100. Additionally, storage 202 stores an activity tracking application 210, that when executed by processor 204, allows a user to interact with the collected biometric information.

In various embodiments, a user may interact with activity tracking application 210 via a GUI 205 including a display 206, such as, for example, a touchscreen display that accepts various hand gestures as inputs. In accordance with various embodiments, activity tracking application 210 may process the biometric information collected by earphones 100 and present it via display 206 of GUI 205. Before describing activity tracking application 210 in further detail, it is worth noting that in some embodiments earphones 100 may filter the collected biometric information prior to transmitting the biometric information to computing device 200. Accordingly, although the embodiments disclosed herein are described with reference to activity tracking application 210 processing the received biometric information, in various implementations various preprocessing operations may be performed by a processor 160, 165 of earphones 100.

In various embodiments, activity tracking application 210 may be initially configured/setup (e.g., after installation on a smartphone) based on a user's self-reported biological information, sleep information, and activity preference information. For example, during setup a user may be prompted via display 206 for biological information such as the user's gender, height, age, and weight. Further, during setup the user may be prompted for sleep information such as the amount of sleep needed by the user and the user's regular bed time. Further, still, the user may be prompted during setup for a preferred activity level and activities the user desires to be tracked (e.g., running, walking, swimming, biking, etc.) In various embodiments, described below, this self-reported information may be used in tandem with the information collected by earphones 100 to display activity monitoring information using various modules.

Following setup, activity tracking application 210 may be used by a user to monitor and define how active the user wants to be on a day-to-day basis based on the biometric information (e.g., accelerometer information, optical heart rate sensor information, etc.) collected by earphones 100. As illustrated in FIG. 4B, activity tracking application 210 may comprise various display modules, including an activity display module 211, a sleep display module 212, an activity recommendation and fatigue level display module 213, and a biological data and intensity recommendation display module 214. Additionally, activity tracking application 210 may comprise various processing modules 215 for processing the activity monitoring information (e.g., optical heartrate information, accelerometer information, gyroscope information, etc.) collected by the earphones or the biological information entered by the users. These modules may be implemented separately or in combination. For example, in some embodiments activity processing modules 215 may be directly integrated with one or more of display modules 211-214.

As will be further described below, each of display modules 211-214 may be associated with a unique display provided by activity tracking app 210 via display 206. That is, activity display module 211 may have an associated activity display, sleep display module 212 may have an associated sleep display, activity recommendation and fatigue level display module 213 may have an associated activity recommendation and fatigue level display, and biological data and intensity recommendation display module 214 may have an associated biological data and intensity recommendation display.

FIG. 4C is a schematic block diagram illustrating exemplary processing modules embodied in the disclosed computing device in accordance with embodiments of the disclosed technology. Computing device embodies processing modules 215 for providing a smart activity score 215 with movement monitoring module 218, period activity score module 219, and smart activity score module 217.

Movement monitoring module 218 monitors a movement to determine a metabolic loading associated with the movement during a score period. Movement monitoring module 218 will be described below in further detail with regard to various processes.

Period activity score module 219 creates and updates a period activity score based on the metabolic loading and the movement. The period activity score is created and updated for the score period. Period activity score module 219 will be described below in further detail with regard to various processes.

In one embodiment, a fatigue level module (not shown) detects a fatigue level. The fatigue level module will be described below in further detail with regard to various processes.

Smart activity score module 217 creates and updates a smart activity score by aggregating a set of period activity scores. Smart activity score module 217 will be described below in further detail with regard to various processes.

FIG. 4D is a schematic block diagram illustrating further exemplary processing modules 215 embodied in the disclosed computing device for providing a smart activity score with movement monitoring module 218, period activity score module 219, and smart activity score module 217. Computing device also includes user lifestyle module 216 and period activity score multiplier module 220. User lifestyle module 216 and period activity score multiplier module 220 will be described below in further detail with regard to various processes.

Although FIGS. 4C-4D depict modules as processing modules 215 of computing device 200, the processing modules may also be embodied in a wearable sensor. In particular, in various embodiments, at least one of movement monitoring module 218, period activity score module 219, smart activity score module 218, user lifestyle module 216, and period activity score multiplier module 220 is embodied in a wearable sensor, such as earphones 100 disclosed herein. Moreover, any of the modules described herein may be embodied in earphones 100 or in other hardware or devices. Any of the modules described herein may connect to other modules described herein via any wired or wireless communication medium, such as those described in connection with communication link 300.

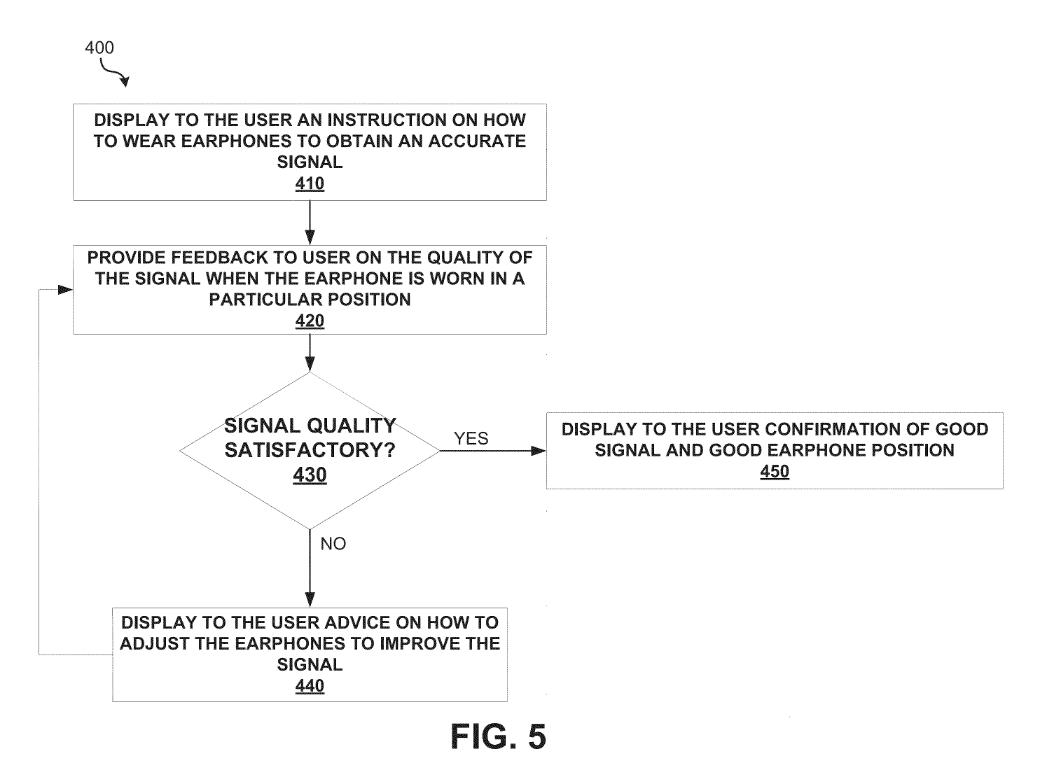

In embodiments, activity tracking application 210 may be used to display to the user an instruction for wearing and/or adjusting earphones 100 if it is determined that optical heartrate sensor 122 and/or motion sensor 121 are not accurately gathering motion data and heart rate data. FIG. 5 is an operational flow diagram illustrating one such method 400 of an earphone adjustment feedback loop with a user that ensures accurate biometric data collection by earphones 100. At operation 410, execution of application 210 may cause display 206 to display an instruction to the user on how to wear earphones 100 to obtain an accurate and reliable signal from the biometric sensors. In embodiments, operation 410 may occur once after installing application 210, once a day (e.g., when user first wears the earphones 100 for the day), or at any custom and/or predetermined interval.

At operation 420, feedback is displayed to the user regarding the quality of the signal received from the biometric sensors based on the particular position that earphones 100 are being worn. For example, display 206 may display a signal quality bar or other graphical element. At decision 430, it is determined if the biosensor signal quality is satisfactory for biometric data gathering and use of application 210. In various embodiments, this determination may be based on factors such as, for example, the frequency with which optical heartrate sensor 122 is collecting heart rate data, the variance in the measurements of optical heartrate sensor 122, dropouts in heart rate measurements by sensor 122, the signal-to-noise ratio approximation of optical heartrate sensor 122, the amplitude of the signals generated by the sensors, and the like.

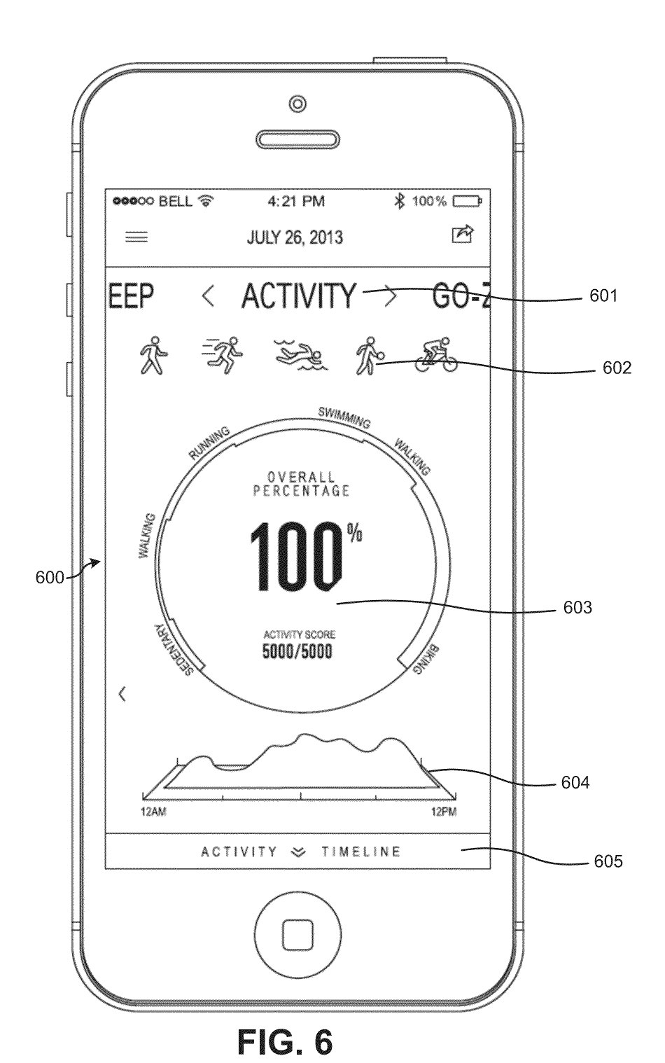

If the signal quality is unsatisfactory, at operation 440, application 210 may cause display 206 to display to the user advice on how to adjust the earphones to improve the signal, and operations 420 and decision 430 may subsequently be repeated. For example, advice on adjusting the strain relief of the earphones may be displayed. Otherwise, if the signal quality is satisfactory, at operation 450, application may cause display 206 to display to the user confirmation of good signal quality and/or good earphone position. Subsequently, application 210 may proceed with normal operation (e.g., display modules 211-214). FIGS. 6, 9-11 illustrate a particular exemplary implementation of a GUI for application 210 comprising displays associated with each of display modules 211-214.

FIG. 6 illustrates an activity display 600 that may be associated with an activity display module 211. In various embodiments, activity display 600 may visually present to a user a record of the user's activity. As illustrated, activity display 600 may comprise a display navigation area 601, activity icons 602, activity goal section 603, live activity chart 604, and activity timeline 605. As illustrated in this particular embodiment, display navigation area 601 allows a user to navigate between the various displays associated with modules 211-214 by selecting “right” and “left” arrows depicted at the top of the display on either side of the display screen title. An identification of the selected display may be displayed at the center of the navigation area 601. Other selectable displays may displayed on the left and right sides of navigation area 601. For example, in this embodiment the activity display 600 includes the identification “ACTIVITY” at the center of the navigation area. If the user wishes to navigate to a sleep display in this embodiment, the user may select the left arrow. In implementations where device 200 includes a touch screen display, navigation between the displays may be accomplished via finger swiping gestures. For example, in one embodiment a user may swipe the screen right or left to navigate to a different display screen. In another embodiment, a user may press the left or right arrows to navigate between the various display screens.

In various embodiments, activity icons 602 may be displayed on activity display 600 based on the user's predicted or self-reported activity. For example, in this particular embodiment activity icons 602 are displayed for the activities of walking, running, swimming, sport, and biking, indicating that the user has performed these five activities. In one particular embodiment, one or more modules of application 210 may estimate the activity being performed (e.g., sleeping, walking, running, or swimming) by comparing the data collected by a biometric earphone's sensors to pre-loaded or learned activity profiles. For example, accelerometer data, gyroscope data, heartrate data, or some combination thereof may be compared to preloaded activity profiles of what the data should look like for a generic user that is running, walking, or swimming. In implementations of this embodiment, the preloaded activity profiles for each particular activity (e.g., sleeping, running, walking, or swimming) may be adjusted over time based on a history of the user's activity, thereby improving the activity predictive capability of the system. In additional implementations, activity display 600 allows a user to manually select the activity being performed (e.g., via touch gestures), thereby enabling the system to accurately adjust an activity profile associated with the user-selected activity. In this way, the system's activity estimating capabilities will improve over time as the system learns how particular activity profiles match an individual user. Particular methods of implementing this activity estimation and activity profile learning capability are described in U.S. patent application Ser. No. 14/568,835, filed Dec. 12, 2014, titled “System and Method for Creating a Dynamic Activity Profile”, and which is incorporated herein by reference in its entirety.

In various embodiments, the percentage activity goal may be selected by the user (e.g., by a touch tap) to display to the user an amount of a particular activity (e.g., walking or running) needed to complete the activity goal (e.g., reach 100%). In additional embodiments, activities for the timeframe may be individually selected to display metrics of the selected activity such as points, calories, duration, or some combination thereof. For example, in this particular embodiment activity goal section 603 displays that 100% of the activity goal for the day has been accomplished. Further, activity goal section 603 displays that activities of walking, running, biking, and no activity (sedentary) took place during the day. This is also displayed as a numerical activity score 5000/5000. In this embodiment, a breakdown of metrics for each activity (e.g., activity points, calories, and duration) for the day may be displayed by selecting the activity.

A live activity chart 604 may also display an activity trend of the aforementioned metrics (or other metrics) as a dynamic graph at the bottom of the display. For example, the graph may be used to show when user has been most active during the day (e.g., burning the most calories or otherwise engaged in an activity).

An activity timeline 605 may be displayed as a collapsed bar at the bottom of display 600. In various embodiments, when a user selects activity timeline 605, it may display a more detailed breakdown of daily activity, including, for example, an activity performed at a particular time with associated metrics, total active time for the measuring period, total inactive time for the measuring period, total calories burned for the measuring period, total distance traversed for the measuring period, and other metrics.

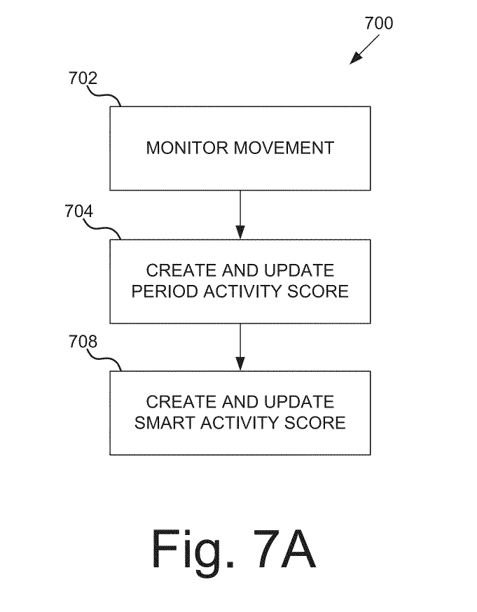

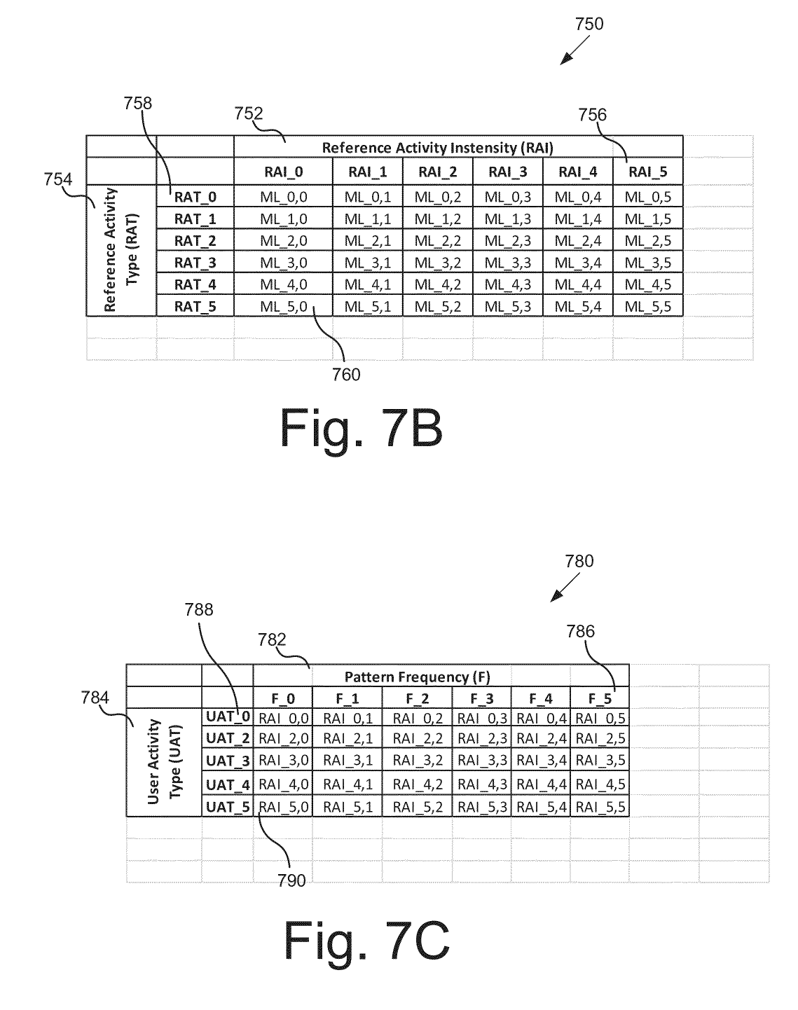

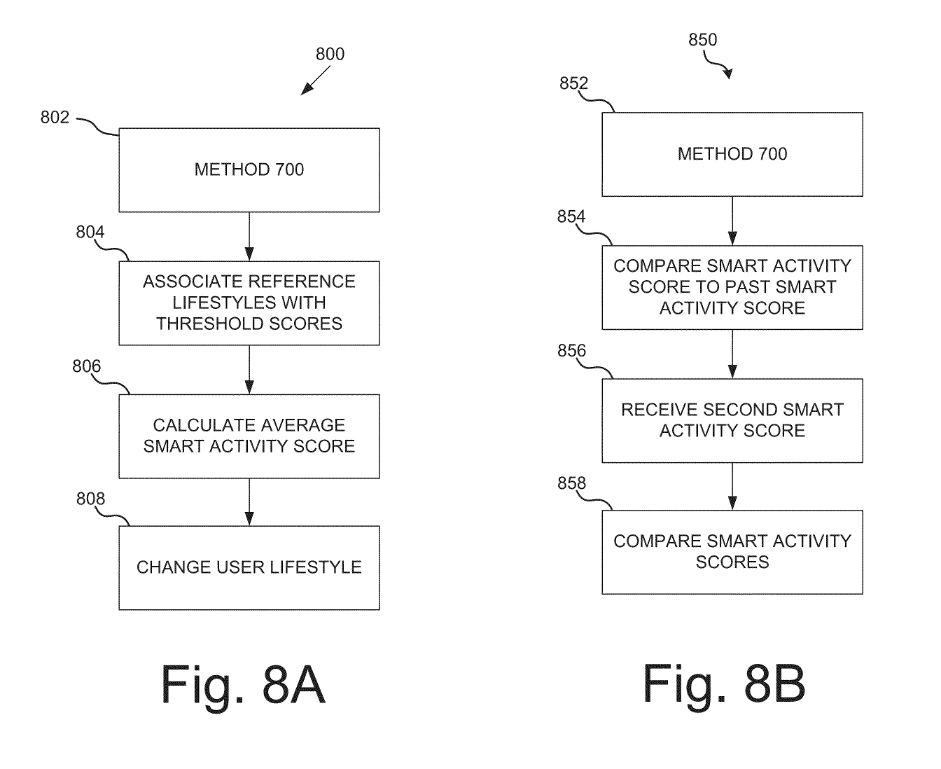

In various embodiments, an activity goal section 603 may display various activity metrics such as a percentage activity goal providing an overview of the status of an activity goal for a timeframe (e.g., day or week), an activity score or other smart activity score associated with the goal, and activities for the measured timeframe (e.g., day or week). For example, the display may provide a user with a current activity score for the day versus a target activity score for the day. Particular methods of calculating activity scores are described in U.S. patent application Ser. No. 14/137,734, filed Dec. 20, 2013, titled “System and Method for Providing a Smart Activity Score”, and which is incorporated herein by reference in its entirety. Some particular methods for calculating and providing smart activity scores described in connection with FIGS. 7A-7C and 8A-8B.

For example, FIG. 7A is an operational flow diagram illustrating an example of a method 700 for creating and updating a smart activity score in accordance with an embodiment of the present disclosure. The operations of method 700 create and update a smart activity score based on a user's activity. Moreover, the operations of method 700 take into account changes that occur in user activities as they occur in short time segments. This provides high-resolution activity monitoring. In an additional embodiment, metabolic loadings are tailored to the specific characteristics of the user. This provides for increased accuracy in tracking the user's activity levels. In one embodiment, earphones 100, and computing device 200 perform various operations of method 700.

At operation 702, method 700 monitors a movement to determine a metabolic loading associated with the movement during a score period. In one embodiment, the metabolic loadings are determined by identifying a user activity type from a set of reference activity types and by identifying a user activity intensity from a set of reference activity intensities.

Method 700, in one embodiment, determines a set of metabolic loadings according to information provided by a user (or user information). User information may include, for example, an individual's height, weight, age, gender, and geographic and environmental conditions. The user may provide the user information by, for example, a user interface 205 of computing device 200, or controller 130 of earphones 100. Method 700 may determine the user information based on various measurements. For example, method 700 may determine a user's body fat content or body type. Or, for example, method 700 may use an altimeter or GPS embodied in either the computing device 200 or earphones 11 to determine the user's elevation, weather conditions in the user's environment, etc. In another embodiment, method 700 obtains user information from the user indirectly. For example, method 700 may collect user information from a social media account, from a digital profile, or the like.

In one embodiment, the user information includes a user lifestyle selected from a set of reference lifestyles. At operation 702, method 700 prompts the user for information about the user's lifestyle (e.g., via user interface 205 of computing device 200, or via earphones 100). Method 700 may prompt the user to determine how active the user's lifestyle is. For example, method 700 may prompt the user to select a user lifestyle from a set of reference lifestyles. In one embodiment, the reference lifestyles include a range of lifestyles from inactive, on one end, to highly active on the other end. So, for example, the reference lifestyles that the user may select from may include sedentary, mildly active, moderately active, and heavily active.

At operation 702, method 700, in one embodiment, determines the user lifestyle from the user as an initial matter. In a further embodiment, method 700 periodically prompts the user to select a user lifestyle. In this fashion, the user lifestyle selected may be aligned with the user's actual activity level as the user's activity level varies over time. In a further embodiment, method 700 updates the user lifestyle without intervention from the user.

In one embodiment, the metabolic loadings are numerical values and may represent a rate of calories burned per unit weight per unit time (e.g., having units of kcal per kilogram per hour). By way of example, the metabolic loadings can be represented in units of oxygen uptake (e.g., in milliliters per kilogram per minute). The metabolic loadings may also represent a ratio of the metabolic rate during activity (e.g., the metabolic rate associated with a particular activity type and/or an activity intensity) to the metabolic rate during rest. The metabolic loadings, may, for example, be represented in a metabolic table, such as metabolic table 750 in FIG. 7B. In one embodiment, the metabolic loadings are specific to the user information. For example, a metabolic loading may increase for a heavier user, or for an increased elevation, but may decrease for a lighter user or for a decreased elevation.

At operation 702, in one embodiment, method 700 determines the set of metabolic loadings based on the user lifestyle, in addition to the other user information. For example, the metabolic loadings for a user with a heavily active lifestyle may differ from the metabolic loadings for a user with a sedentary lifestyle. Method 700 may attain greater coupling between the metabolic loadings and the user's characteristics by determining the set of metabolic loadings according to the user lifestyle.

In various embodiments, a device (e.g., computing device 200, earphones 100) or a module (e.g. modules embodied in activity tracking application 210) stores or provides the metabolic loadings. The metabolic loadings may be maintained or provided by a server or over a communication medium such as a medium used in connection with communication link 300. In one embodiment, a system administrator provides the metabolic loading based on a survey, publicly available data, scientifically determined data, compilation of user data, or any other source of data. Operation 702, in various embodiments, is performed by movement monitoring module 218. In various embodiments, movement monitoring module 218 includes a metabolic loading module and a metabolic table module that determine the metabolic loading associated with the movement.

At operation 702, method 700, in one embodiment, maintains a metabolic table based on the user information. For example, the metabolic loadings in the metabolic table may be based on the user information from the user. In some cases, the metabolic table is maintained based on a set of standard user information, in place of or in addition to user information from the user. The standard user information may include, for example, the average fitness characteristics of all individuals being the same age as the user, the same height as the user, etc. In another embodiment, instead of maintaining the metabolic table based on standard information, if method 700 has not obtained user information from the user, method 700 delays maintaining the metabolic table until the user information is obtained.

As illustrated in FIG. 7B at operation 702, in one embodiment, method 700 maintains the metabolic table as metabolic table 750. Metabolic table 750 may be stored in computing device 200, for example. Metabolic table 750 may include information such as reference activity types (RATs) 754, reference activity intensities (RAIs) 752, and/or metabolic loadings (MLs) 760.

In one embodiment, RATs 754 are arranged as rows 758 in metabolic table 750. Thus, each of a set of rows 758 corresponds to different RATs 754, and each row 758 is designated by a row index number. For example, the first RAT row 758 may be indexed as RAT_0, the second as RAT_1, and so on for as many rows as metabolic table 750 may include.

The reference activity types may include typical activities, such as running, walking, sleeping, swimming, bicycling, skiing, surfing, resting, working, and so on. The reference activity types may also include a catch-all category, for example, general exercise. The reference activity types may also include atypical activities, such as skydiving, SCUBA diving, and gymnastics. In one embodiment, a user defines a user-defined activity by programming computing device 200 (e.g., via an interface 205 on computing device 200) with information about the user-defined activity, such as pattern of movement, frequency of pattern, and intensity of movement. The typical reference activities may be provided, for example, by metabolic table 750.

In one embodiment, reference activity intensities 752 are arranged as columns 756 in metabolic table 750, and metabolic table 750 includes columns 756, each corresponding to different RAIs 752. Each column 756 is designated by a different column index number. For example, the first RAI column 756 may be indexed as RAI_0, the second as RAI_1 and so on for as many columns as metabolic table 750 may include.

The reference activity intensities, in one illustrative case, include a numeric scale. For example, the reference activity intensities may include numbers ranging from one to ten (representing increasing activity intensity). The reference activities may also be represented as a range of letters, colors, and the like. The reference activity intensities may be associated with the vigorousness of an activity. In other embodiments, the reference activity intensity are represented by ranges of heart rates or breathing rates.

In one embodiment, metabolic table 750 includes metabolic loadings, such as metabolic loading 760. Each metabolic loading 760 corresponds to a reference activity type 758 of the reference activity types 754 and a reference activity intensity 756 of the reference activity intensities 752. Each metabolic loading 760 may be identified by a unique combination of reference activity type 754 and reference activity intensity 752. For example, in the column and row arrangement discussed above, one of the reference activity types 754 of a series of rows 758 of reference activity types, and one of the reference activity intensities 752 of a series of columns 756 of reference activity intensities may correspond to a particular metabolic loading 760. In such an arrangement, each metabolic loading 760 may be identifiable by only one combination of reference activity type 758 and reference activity intensity 756.

This concept is illustrated in FIG. 7B. As shown, each metabolic loading 760 is be designated using a two-dimensional index, with the first index dimension corresponding to the row 758 number and the second index dimension corresponding to the column 756 number of the metabolic loading 760. For example, in FIG. 7B, ML_2,3 has a first dimension index of 2 and a second dimension index of 3. ML_2,3 corresponds to the row 758 for RAT_2 and the column 756 for RAI_3. Any combination of RAT_M and RAI_N may identify a corresponding ML_M,N in metabolic table 750, where M is any number corresponding to a row 758 number in metabolic table 750 and N is any number corresponding to a column 756 number in metabolic table 750. For example, the reference activity type RAT_3 may be “surfing,” and the reference activity intensity RAI_3 may be “4.” This combination in the metabolic table 750 corresponds to metabolic loading 760 ML_3,3, which may, for example, represent 5.0 kcal/kg/hour (a typical value for surfing). In various embodiments, operation 702 is performed by movement monitoring module 218. In some embodiments, operation 702 is performed by a metabolic table module.

Referring again to operation 702 of method 700, in one embodiment, the movement is monitored by location tracking (e.g., Global Positioning Satellites (GPS), or a location tracking device connected via a communications medium similar to that used for communications link 300). In some instances, the general location of the user as well as specific movements of the user's body are monitored. For example, method 700 may monitor the movement of the user's leg in x, y, and z directions (e.g., by an accelerometer or gyroscope). In one embodiment, method 700 receives an instruction regarding which body part is being monitored. For example, method 700 may receive an instruction that the movement of a user's wrist, ankle, head, or torso is being monitored. For example, when the earphones 100 of the present disclosure are being worn, an instruction that the user's head movement is being monitored may be provided. In another example, although the earphones are being worn, the user may also be carrying computing device 200 in a hand or in an attachment on an arm or leg. In this example, an instruction that the user's hand, arm or leg movement is being monitored may be provided in lieu of, or in combination with, movement detected by the earphones 100.

Method 700, in various embodiments, monitors the movement of the user and determines a pattern of the movement (pattern). For example, method 700 may detect the pattern by an accelerometer or gyroscope embodied in earphones 100 or computing device 200. The pattern may be a repetition of a motion or a similar motion monitored at operation 702. In one embodiment, the pattern is a geometric shape (e.g., a circle, line, oval) of repeated motion that is monitored. In some cases, the repetition of a motion in a geometric shape is not repeated consistently over time, but is maintained for a substantial proportion of the repetitions of movement. For example, one occurrence of elliptical motion in a repetitive occurrence (or pattern) of ten circular motions may be monitored and determined to be a pattern of circular motion.

In further embodiments, the geometric shape of the pattern of movement is a three dimensional (3-D) shape. For example, the pattern of movement associated with the arm, hand, wrist or head of a person running a marathon may be monitored and analyzed into a geometric shape in three dimensions. The pattern may be complicated, but it may be described in a form that method 700 can recognize when performing operation 702. Such form may include computer code that describes the spatial relationship of a set of points, along with changes in acceleration forces that are experienced along those points as, for example, a sensor travels throughout the pattern.

At operation 702, in some instances, monitoring the pattern includes monitoring the frequency with which the pattern is repeated (or pattern frequency). The pattern frequency may be derived from a repetition period of the pattern (or pattern repetition period). The pattern repetition period may be the length of time elapsing from when a device or sensor passes through a certain point in a pattern and when the device or sensor returns to that point when the pattern is repeated. For example, the sensor may be at point x, y, z at time t_0. The device may then move along the trajectory of the pattern, eventually returning to point x, y, z at time t_1. The pattern repetition period would be the difference between t_1 and t_0 (e.g., measured in seconds). For example, method 700 may determine that the pattern is a circle and that the circle pattern repetition period is one second. The pattern frequency may be the inverse of the pattern repetition period, and may have units of cycles per second. When the pattern repetition period is, for example, two seconds, the pattern frequency would be 0.5 cycles per second.

In various embodiments, monitoring the movement at operation 702 includes monitoring the velocity at which the user is moving (or the user velocity). For example, the user velocity may have units of kilometers per hour. Method 700, in one embodiment, monitors the user's location information to determine user velocity. Method 700 may do this by GPS, through communication medium 704, and so on. The user velocity may be distinguished from the speed of the pattern (or pattern speed). For example, the user may be running at a user velocity 10 km/hour, but the pattern speed of the user's hand (e.g. carrying the computing device) may be 20 km/hour at a given point (e.g., as the hand moves from behind the user to in front of the user). At operation 702, the pattern speed may be monitored using, for example, an accelerometer or gyroscope. In still a further example, the user velocity may also be distinguished from the speed of the pattern (or pattern speed) of a user's head (e.g., as the head rocks slightly forward and backward when running or jogging), although slight, as it is monitored by sensors embodied in earphones 100.

At operation 702, in one embodiment, method 700 monitors the user's altitude. This may be done, for example, using an altimeter embodied in computing device 200 or earphones 100. Method 700 may do this using other means, such as use location information, information entered by the user, etc. In another embodiment, at operation 702, method 700 monitors an impact the user has with an object. For example, method 700 may monitor the impact of the user's feet with ground. Method 700 may do this using, for example, an accelerometer or gyroscope.

In various embodiments, method 700 measures the ambient temperature. For example, method 700 may associate a group of reference activity types with bands of ambient temperature. For example, when the ambient temperature is zero degrees Celsius, activities such as skiing, sledding, and ice climbing are appropriate selections for reference activity types, whereas surfing, swimming, and beach volleyball may be inappropriate. In further embodiments, the humidity may be measured (e.g., by a hygrometer). In further embodiments, at operation 702, method 700 measures the pattern duration, that is, the length of time for which particular movement pattern is sustained.

Method 700, in some cases, performs operation 702, monitoring the movement, by using a sensor configured to be attached to a user's body. Such sensors may include a gyroscope or accelerometer to detect movement, and a heart-rate sensor, each of which may be embedded in a pair of earphones that a user can situate in his or her ears, such as earphones 100. Additionally, various modules and sensors that may be used to perform operation 702 may be embedded in a computing device, such as computing device 200 or earphones 100. In various embodiments, operation 702 is performed by movement monitoring module 218.

In one embodiment, operation 702 involves determining the user activity type from the set of reference activity types. Once detected, the pattern may be used to determine the user activity type from a set of reference activity types. In one illustrative instance, each reference activity type is associated with a reference activity type pattern. The user activity type may be determined to be the reference activity type that has a reference activity type pattern that matches the pattern measured at operation 702. In one embodiment, the pattern that matches the reference activity type pattern will not be an exact match, but will be substantially similar.

The patterns, in other embodiments, will not even be substantially similar, but method 700 will determine that the patterns match because they are the most similar of any patterns available. For example, the reference activity type may be determined such that the difference between the pattern of movement corresponding to this reference activity type and the pattern of movement is less than a predetermined range or ratio. In one embodiment, the pattern is looked up (for a match) in a reference activity type library. The reference activity type library may be included in the metabolic table. For example, the reference type library may include rows in a table such as the RAT rows 758.

In further embodiments, operation 702 involves using the pattern frequency to determine the user activity type from the set of reference activity types. For example, several reference activity types may be associated with similar patterns (e.g., because the wrist moves in a similar pattern when running versus walking). Method 700 may measure a pattern and not be able to determine whether the corresponding user activity type is walking or running. Method 700 may use the pattern frequency to determine the activity type in such an example because the pattern frequency for running may be higher than the pattern frequency for walking.

Operation 702 involves, in one embodiment, using additional information to determine the activity type of the user. For example, the pattern for walking may be similar to the pattern for running. Method 700 may associate the reference activity of running with higher user velocities and may associate the reference activity of walking with lower user velocities. Method 700 may use the velocity measured at operation 702 to determine between two reference activity types having similar patterns.

In another embodiment, operation 702 involves monitoring the impact the user has with the ground, and determine that, because the impact is larger, the activity type is running rather than walking. Moreover, if there is no impact, method 700 may determine that the activity type is cycling (or other activity where there is no impact). In another embodiment, method 700 uses a temperature measurement to narrow the reference activity types to those that are performed in the summer, winter, or the like, and uses that information to determine which activity is being performed. For example, the method may have narrowed the possible activities to snow-skiing or water-skiing based on factors other than temperature. Method 700 may then use the temperature measurement to determine that the activity being performed is snow-skiing rather than water-skiing because the temperature is measured to be 0° Celsius.

Operation 702, in another case, entails instructing the user to confirm the user activity type. For example, user interface 205 associated with activity tracking application 210 may allow the user to confirm whether a displayed user activity type is correct. In another embodiment, a user interface 215 associated with the activity tracking application 210 allows the user to select the user activity type from a group of activity types.

In further embodiments, at operation 702, method 700 determines a statistical likelihood for of choices for user activity type and provide the possible user activity types in such a sequence that the most likely user activity type is listed first (and then in descending order of likelihood). For example, method 700 may detect a pattern of movement and determine that, based on the pattern, the pattern frequency, the temperature, and so on, there is an 80% chance the user activity type is running, a 15% chance the user activity type is walking, and a 5% chance the user activity is dancing. Method 700 may then, via a user interface, list these possible user activities such that the user may select the activity type the user is performing. In various embodiments, portions of operation 702 are performed by a metabolic loading module.

At operation 702, in one embodiment, method 700 determines the user activity intensity from a set of reference activity intensities. Method 700 may determine the user activity intensity in a variety of ways. In one embodiment, method 700 associates the repetition period (or pattern frequency) and user activity type (UAT) with a reference activity intensity library to determine the user activity intensity that corresponds to a reference activity intensity. FIG. 7C illustrates one embodiment whereby this aspect of operation 702 is accomplished, including reference activity intensity library 780. Library 780 is organized by rows 788 of reference activity types 784 and columns 786 of pattern frequencies 782. In FIG. 7C, library 780 is implemented in a table. Library 780 may, however, be implemented other ways.

In one embodiment, at operation 702, method 700 determines that, for user activity type 784 UAT_0 performed at pattern frequency 782 F_0, the reference activity intensity 790 is RAI_0,0. For example, method 700 may determine that UAT 784 corresponds to the reference activity type for running. Method 700 may also determine a pattern frequency 782 of 0.5 cycles per second for the user activity type. Reference activity intensity library 780 may determine, at operation 702, that the user activity type 784 of running at a pattern frequency 782 of 0.5 cycles per second corresponds to a reference activity intensity 790 of five on a scale of ten. In another embodiment, the reference activity intensity 790 is independent of the activity type. For example, method 700 may determine that the repetition period is five seconds, and that this corresponds to an intensity level of two on a scale of ten.

Reference activity intensity library 780, in one embodiment, is included in metabolic table 750. In some cases, the measured repetition period (or pattern frequency) does not correspond exactly to a repetition period for a reference activity intensity in metabolic table 750. In such an example, the correspondence may be a best-match fit, or may be a fit within a tolerance. Such a tolerance may be defined by the user or by a system administrator, for example.

In various embodiments, operation 702 involves supplementing the measurement of pattern frequency to help determine the user activity intensity from the reference activity intensities. For example, if the user activity type is skiing, it may be difficult to determine the user activity intensity because the pattern frequency may be erratic or otherwise immeasurable. In such an example, method 700 may monitor the user velocity, the user's heart rate, and other indicators (e.g., breathing rate) to determine how hard the user is working during the activity. For example, higher heart rate may indicate higher activity intensity. In a further embodiment, the reference activity intensity are associated with a pattern speed (i.e., the speed or velocity at which the sensor is progressing through the pattern). A higher pattern speed may correspond to a higher user activity intensity.

Method 700, in some instances, performs operation 702 to determine the user activity type and the user activity intensity by using a sensor configured to be attached to the user's body (e.g. earphones 100). Such sensors may include, for example, a gyroscope or accelerometer to detect movement (e.g. motion sensor 121 of earphones 100), and a heart-rate sensor (e.g. optical heartrate sensor 122 of earphones 100), each of which may be embedded in a pair of earphones that can be worn in the user's ears, such as earphones 100. Additionally, various sensors and modules that may be used to preform operation 702 may be embedded in computing device 200. In various embodiments, operation 702 is performed by movement monitoring module 218.

Referring again to FIG. 7A, operation 704 includes creating and updating a period activity score based on the metabolic loading and the movement. The period activity score is created and updated for the score period. In one embodiment, method 700 (e.g., at operation 702) determines a duration of the activity type at a particular activity intensity (e.g., in seconds, minutes, or hours). Method 700 may create and update the period activity score by multiplying the metabolic loading by the duration of the user activity type at a particular user activity intensity. If the user activity intensity changes, method 700 may multiply the new metabolic loading (associated with the new user activity intensity) by the duration of the user activity type at the new user activity intensity. Accordingly, in one embodiment, the period activity score is represented as a numerical value.

Additionally, at operation 704, method 700 creates and updates the period activity score based on score periods. In one embodiment, at operation 702, monitoring the movement includes determining, during a score period, the metabolic loading associated with the movement. Score periods may include segments of time. For example, a score period may be ten seconds. For the illustrative score period of ten seconds, each twenty-four day would include 8,640 score periods. In one embodiment, method 700 monitors the user's movement (e.g., at operation 702) to determine a user activity type, a user activity intensity, and a corresponding metabolic loading during each score period. Method 700 may then calculate the period activity score for that score period. As the movement changes over time, the varying characteristics of the movement are captured by the score periods.

In one embodiment, operation 704 includes creating and updating a set of periodic activity scores. Each period activity is based on the movement monitored during a set of score periods, and each period activity score is associated with a particular score period of the set of score periods. In one embodiment, creating and updating the smart activity score includes aggregating a set of period activity scores. The smart activity score may represent a running sum total of the period activity scores.

Operation 704, in one embodiment, includes applying a score period multiplier to the score period to create an adjusted period activity score. In such an embodiment, the smart activity score includes an aggregation of adjusted period activity scores. For example, method 700 may introduce score period multipliers associated with certain score periods, such that the certain score periods contribute more or less to the period activity score than other score periods during which the same movement is monitored. For example, if the user is performing a sustained activity, method 700 may apply a score period multiplier to the score periods that occur during the sustained activity. By contrast, method 700 may not apply a multiplier to score periods that are part of intermittent, rather than sustained, activity. As a result of the score period multiplier, the user's sustained activity may contribute more to the metabolic activity score than the user's intermittent activity. The score period multiplier may allow method 700 to account for the increased demand of sustained, continuous activity relative to intermittent activity.

In one embodiment, the score period multiplier is directly proportional to the number of continuous score periods over which a type and intensity of the movement is maintained. The adjusted period activity score may be greater than or less than the period activity score, depending on the score period multiplier. For example, for intermittent activity, the score period multiplier may be less than 1.0, whereas for continuous, sustained activity, the score period multiplier may be greater than 1.0.

The score period multiplier, in a further embodiment, is directly proportional to the smart activity score for the current measuring period. For example, a user who already has a smart activity score of 2,000 will receive a greater period activity score for going running that a user who has a smart activity score of 1,000. In this way, method 700 may allocate greater points for highly active days relative to moderately active days.

In one embodiment, operation 704 entails decreasing the smart activity score when the user consumes calories. For example, if the user goes running and generates an activity score of 1,000 as a result, but then the user consumes calories, method 700 may decrease the smart activity score by 200 points, or any number of points. The decrease in the number of points may be proportional to the number of calories consumed. In other embodiments, method 700 obtains information about specific aspects of the user's diet, and awards metabolic activity score points for healthy eating (e.g., fiber) and subtracts points for unhealthy eating (e.g., excessive fat consumption).

Method 700, in one embodiment, pushes the user to work harder, or not as hard, depending on the user lifestyle. Method 700 may do this, for example by adjusting the metabolic loadings based on the user lifestyle. For example, a user with a highly active lifestyle may be associated with metabolic loadings that result in a lower metabolic activity score when compared to a user with a less active lifestyle performing the same movements. This results in method 700 requiring the more active user to, for example, work (or perform movement) at a higher activity intensity or for a longer duration to achieve the same metabolic activity score as the less active user participating in the same activity type (or movements).

At operation 704, in one embodiment, the smart activity score is reset every twenty-four hours. Method 700 may continually increment and decrement the smart activity score throughout a measuring period, but may reset the smart activity score to a value (e.g., zero) at the end of twenty-four hours. The smart activity score may be reset after any given length of time (or measuring period). The smart activity score may be continually updated over the period of, for example, one week, or one month.

In one embodiment, method 700 determines that, because the smart activity score was greater than a certain amount for the measuring period, the smart activity score should be reset to a number greater than zero. As such, the user effectively receives a credit for a particularly active day, allowing the user to be less active the next day without receiving a lower smart activity score for the next day. In a further embodiment, method 700 determines that, because the smart activity score was less than a predetermined value for the measuring period, the smart activity score should be reset to a value less than zero. The user effectively receives a penalty for that day, and would have to make up for a particularly inactive, or overly consumptive day by increasing the user's activity levels the next day. In various embodiments, operation 704 is performed by smart activity score module 217.

Method 700, in one embodiment, includes the operation of detecting a fatigue level. In one embodiment, recovery is a function of the fatigue level. In one embodiment, the fatigue level is the fatigue level of the user. Method 700 may detect the fatigue level in various ways. In one embodiment, method 700 detects the fatigue level measuring heart rate variability (HRV) based on the heartrate detected by optical heartrate sensor 122 of earphones 100 (as described in detail in description for FIGS. 2B-3C). For example, when the HRV is determined to be more consistent (i.e., steady, consistent amount of time between heartbeats), the fatigue level may be higher. In other words, the body is less fresh and well-rested. When HRV is more sporadic (i.e., amount of time between heartbeats varies largely), the fatigue level may be lower.

Method 700 may measure HRV in a number of ways. For example, the heart rate variability (HRV) may be measured based on the heartrate information detected by optical heartrate sensor 122 of earphones 100 (as described in detail in description for FIGS. 2B-3C). For example, in one embodiment, method 700 measures the HRV by using the heartrate data gathered from the optical heartrate sensor 122 taking measurements at or near the user's tragus when the earphones 100 are worn.

In one embodiment, method 700 detects the fatigue level based solely on the HRV measured. In a further embodiment, the fatigue level is based on other measurements (e.g., measurements monitored at operation 702) or input from the user (e.g. input via user interface 205 of computing device 200). For example, the fatigue level may be based on the amount of sleep that is measured for the previous night, the amount of sleep that is provided by the user via the user interface 215 of the computing device 200, the duration and type of user activity, and the intensity of the activity that method 700 may determine for a previous time period (e.g., exercise activity level in the last twenty-four hours). By way of example, the factors may include stress-related activities such as work and driving in traffic, which may generally cause a user to become fatigued. In some cases, method 700 detects the fatigue level by comparing the HRV measured to a reference HRV. This reference HRV may be based on information gathered from a large number of people from the general public. In another embodiment, method 700 determines the reference HRV based on past measurements of the user's HRV.

Method 700, in other illustrative instances, detects the fatigue level once every twenty-four hours. This provides information about the user's fatigue level each day so that method 700 may direct the user's activity levels accordingly. In one embodiment, the fatigue level is detected more or less often. Using the fatigue level, a user may determine whether or not an activity is necessary, the appropriate activity intensity, and the appropriate activity duration. For example, in deciding whether to go on a run, or how long to run, the user may want to use method 700 to assess the user's current fatigue level. Then the user may, for example, run for a shorter time if the user is more fatigued, or for a longer time if the user is less fatigued.

Referring again to FIG. 7A, at operation 708, method 700 creates and updates a smart activity score by aggregating a set of period activity scores. In one embodiment, method 700 may create the smart activity score by increasing or decreasing the period activity scores according to the fatigue level. The fatigue level is represented as a numerical value. In one embodiment, the fatigue level is represented as a relative value, for example, as a current fatigue level relative to an average fatigue level for the user. Method 700 may use this relative value to scale, increment, or decrement the period activity scores to create the smart activity score. Thus, the smart activity score may account not only for the movement of the user, but also for the recovery state, or fatigue level, of the user.

In one embodiment, the smart activity score is tuned to the user's fatigue level, and provides information about whether the user is in his or her peak recovery zone. In such an embodiment, by modifying the period activity score based on the fatigue level, operation 708 pushes the user to exercise more or less vigorously (according to the movement's activity type, intensity, or duration) based on various factors that may affect the user's body. Such factors may include sleep amount, stress levels, general lifestyle and health levels, past workout routines, and recent exercise levels. Operation 708, in one instance, involves updating the smart activity score as the period activity scores are aggregated over time (e.g., according to score periods). In one embodiment, operation 708 performs this updating in real time or near real-time. In other cases, the updating is delayed for a period of time.

The smart activity score, in one embodiment, is associated with a measuring period. The smart activity score may be incremented or decremented throughout the measuring period according to the user's movement, including the user activity types and the user activity intensities. In one embodiment, the smart activity score is reset at the end of the measuring period. For example, the smart activity score may be reset to zero or a number other than zero. In another embodiment, the smart activity score is associated with a measuring period that begins when method 700 detects the fatigue level. In one embodiment, the measuring period is twenty-four hours. However, the measuring period may be any amount of time. For example, the measuring period may be one week, one month, and so on, or may be associated with a training schedule for a race or other event. In various embodiments, operation 708 is performed by smart activity score module 217.

FIG. 8A is an exemplary operational flow diagram illustrating one embodiment of method 800 for creating and updating a smart activity score. Method 800 associates reference lifestyles with threshold scores (e.g., at operation 804), calculates an average smart activity score (e.g., at operation 806), and changes a user lifestyle (e.g., at operation 808). Method 800 may also include all the operations of method 700, in some cases.

At operation 804, method 800 associates each reference lifestyle with a lower threshold score and an upper threshold score. In one embodiment, the lower threshold score and the upper threshold score are numerical values. For example, method 800 may associate the sedentary reference lifestyle with a lower threshold score of 1,000 and an upper threshold score of 2,000. In addition, method 800 may associate the mildly active reference lifestyle with a lower threshold score of 2,001 and an upper threshold score of 3,000. The lower threshold score and the upper threshold score associated with each reference lifestyle define a range of threshold scores. In one embodiment, no two ranges of threshold scores overlap. In various embodiments, operation 804 is performed by user lifestyle module 216.

At operation 806, method 800 calculates an average smart activity score from a set of past smart activity scores. Method 800 may calculate the average smart activity score using a mean, median, mode, or other statistical measure. In one embodiment, the average smart activity score is a range that includes a certain number of standard deviations from a mean or median smart activity score. In one instance, each past smart activity score is associated with a past measuring period. In various embodiments, operation 806 is performed by smart activity score module 808.

At operation 808, method 800 changes the user lifestyle to the reference lifestyle associated with the lower threshold score not greater than the average smart activity score and the upper threshold score not less than the average smart activity score. For example, at operation 806, method 800 may calculate the mean smart activity score from each day over the past month to be 3,500 per day, and the user lifestyle may be mildly active. In one embodiment, the mildly active reference lifestyle has a lower and upper threshold score of 2,001 and 3,000, respectively, and the moderately active reference lifestyle has a lower and upper threshold score of 3,001 and 4,000, respectively. Method 800, at operation 808 (in this example), changes the user lifestyle from mildly active to moderately active because the average smart activity score is between 3,001 and 4,000 (i.e., 3,500), the range associated with moderately active. In other embodiments, method 800 includes customizable upper and lower threshold scores for each reference lifestyle. Operation 808, in various embodiments, is performed by smart activity score module 808 or by metabolic table module 750.

FIG. 8B is an exemplary operational flow diagram illustrating one embodiment of method 850 for creating and updating a smart activity score. Method 850 compares the smart activity score to a past smart activity score (e.g., at operation 854), receives a second smart activity score (e.g., at operation 856), and compares the smart activity score to the second smart activity score (e.g., at operation 858).

At operation 854, method 850 compares the smart activity score to a past smart activity score, and the past smart activity score is associated with a past measuring period. In one embodiment, method 850 stores smart activity scores associated with past measuring periods. Method 850 may recall any past smart activity score and use information associated with that past smart activity score to inform the user's current activity.

At operation 854, in one embodiment, method 850 compares the smart activity score to the smart activity score from the past measuring period by providing a simple numerical readout of both scores (e.g., side by side). In one embodiment, method 850 presents information about the time of day associated with the past smart activity score. For example, method 850 indicates that the past smart activity score was at a particular level at a particular time of day. For example, if the current time is 2:00 PM, method 850 may present the information that on the past day of Oct. 12, 2013, the past smart activity score was 1,200 at 2:00 PM. This may inform the user of how the user's current smart activity score is progressing throughout the measuring period in relation to the past smart activity score.

In another embodiment, operation 854 entails displaying a graph (e.g., a line or bar graph)—via the interface 205 associated with the activity tracking application 210 on computing device 200—of the past smart activity score as a function of time in the past measuring period (e.g., activity score on the y-axis and time on the x-axis). Method 850 may overlay that graph with a graph of the current smart activity score as a function of time over the current measuring period. This may inform the user of the progress of the current measuring period's activity in relation to the past measuring period's activity. One of ordinary skill in the art will appreciate other ways that method 850 may compare the smart activity scores at operation 854. In another embodiment, method 850 compares the smart activity score to multiple past smart activity scores associated with past measuring periods. The depiction of past or current smart activity scores in the interface 205 of the activity tracking application 210, in a further embodiment, is broken down by amount contributed per score period. In various embodiments, operation 854 is performed by smart activity score module 808.

At operation 856, method 850 receives a second smart activity score from a second user. Method 850 may receive the second smart activity score in a number of ways. For example, method 850 may receive the second smart activity score via communication medium 704.

The second smart activity score may be created and updated at operation 856 in a manner substantially similar to the creating and updating of the smart activity score at operation 708. In one embodiment, the second smart activity score represents the modified version of a second smart activity score that is modified or adjusted according to a fatigue level of the second user. In another embodiment, the second activity score may be modified by the period activity score multiplier of the second user. The second user may be any user other than the user. For example, the second user may be a friend or associate of the first user. In various embodiments, operation 856 is performed by smart activity score module 808.

At operation 858, method 850 compares the smart activity score to the second smart activity score. Method 850 may compare the smart activity score to the second smart activity score in many of the same ways that method 850 may compare the smart activity score to the past smart activity score (e.g., at operation 854). Method 850 may compare the two scores using overlaid graphs or other visual depictions in the activity tracking application, by using side-by-side numbers, and the like. In one example, this comparison allows the user to compare the user's daily activity level to the daily activity level of another user. In another example, both users' activity levels are tuned to each user's respective fatigue level. The measuring period, however, for the smart activity score and the second smart activity score may be different. In one instance, method 850 takes into account possible different measuring periods for the two smart activity scores, and normalizes the scores to account for this difference. For example, if the second user is on the East Coast, and the user is on the West Coast, method 850 may adjust the smart activity score comparison to account for this difference. In various embodiments, operation 858 is performed by smart activity score module 808.

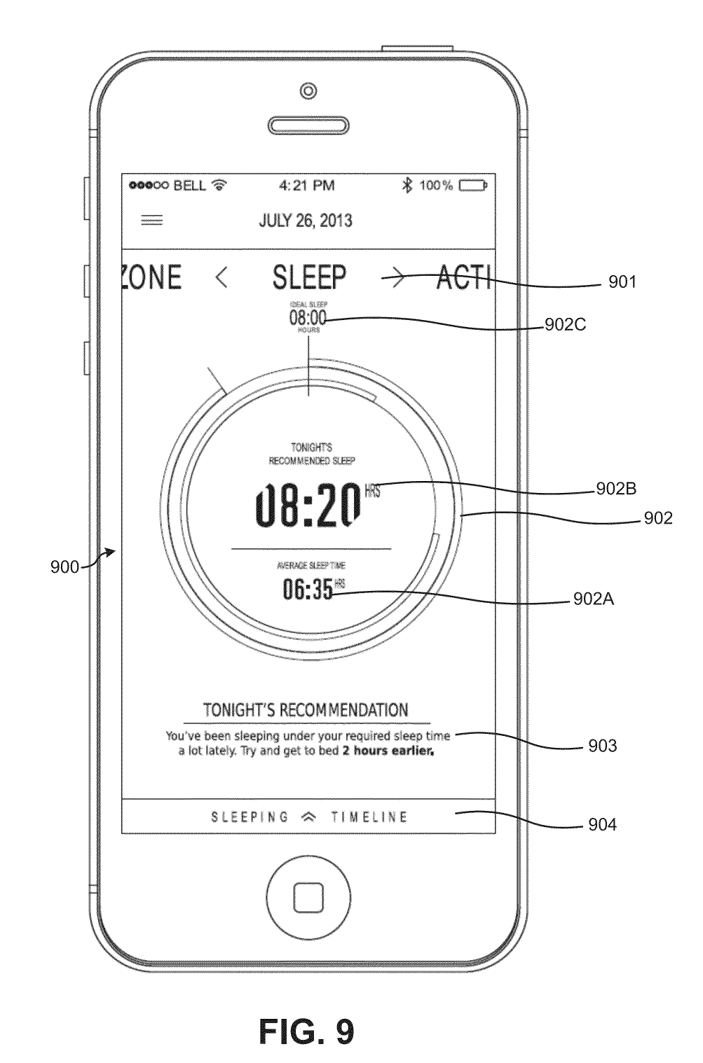

Returning now to a discussion of the various exemplary activity displays associated with display modules 211-214, FIG. 9 illustrates a sleep display 900 that may be associated with a sleep display module 212. In various embodiments, sleep display 900 may visually present to a user a record of the user's sleep history and sleep recommendations for the day. It is worth noting that in various embodiments one or more modules of the activity tracking application 210 may automatically determine or estimate when a user is sleeping (and awake) based on an a pre-loaded or learned activity profile for sleep, in accordance with the activity profiles described above. Alternatively, the user may interact with the sleep display 900 or other display to indicate that the current activity is sleep, enabling the system to better learn that individualized activity profile associated with sleep. The modules may also use data collected from the earphones, including fatigue level and activity score trends, to calculate a recommended amount of sleep. Systems and methods for implementing this functionality are described in greater detail in U.S. patent application Ser. No. 14/568,835, filed Dec. 12, 2014, and titled “System and Method for Creating a Dynamic Activity Profile”, and U.S. patent application Ser. No. 14/137,942, filed Dec. 20, 2013, titled “System and Method for Providing an Interpreted Recovery Score,” both of which are incorporated herein by reference in their entirety.

As illustrated, sleep display 900 may comprise a display navigation area 901, a center sleep display area 902, a textual sleep recommendation 903, and a sleeping detail or timeline 904. Display navigation area 901 allows a user to navigate between the various displays associated with modules 211-214 as described above. In this embodiment the sleep display 900 includes the identification “SLEEP” at the center of the navigation area 901.

Center sleep display area 902 may display sleep metrics such as the user's recent average level of sleep or sleep trend 902A, a recommended amount of sleep for the night 902B, and an ideal average sleep amount 902C. In various embodiments, these sleep metrics may be displayed in units of time (e.g., hours and minutes) or other suitable units. Accordingly, a user may compare a recommended sleep level for the user (e.g., metric 902B) against the user's historical sleep level (e.g., metric 902A). In one embodiment, the sleep metrics 902A-902C may be displayed as a pie chart showing the recommended and historical sleep times in different colors. In another embodiment, sleep metrics 902A-902C may be displayed as a curvilinear graph showing the recommended and historical sleep times as different colored, concentric lines. This particular embodiment is illustrated in example sleep display 900, which illustrates an inner concentric line for recommended sleep metric 902B and an outer concentric line for average sleep metric 902A. In this example, the lines are concentric about a numerical display of the sleep metrics.

In various embodiments, a textual sleep recommendation 903 may be displayed at the bottom or other location of display 900 based on the user's recent sleep history. A sleeping detail or timeline 904 may also be displayed as a collapsed bar at the bottom of sleep display 900. In various embodiments, when a user selects sleeping detail 904, it may display a more detailed breakdown of daily sleep metrics, including, for example, total time slept, bedtime, and wake time. In particular implementations of these embodiments, the user may edit the calculated bedtime and wake time. In additional embodiments, the selected sleeping detail 904 may graphically display a timeline of the user's movements during the sleep hours, thereby providing an indication of how restless or restful the user's sleep is during different times, as well as the user's sleep cycles. For the example, the user's movements may be displayed as a histogram plot charting the frequency and/or intensity of movement during different sleep times.

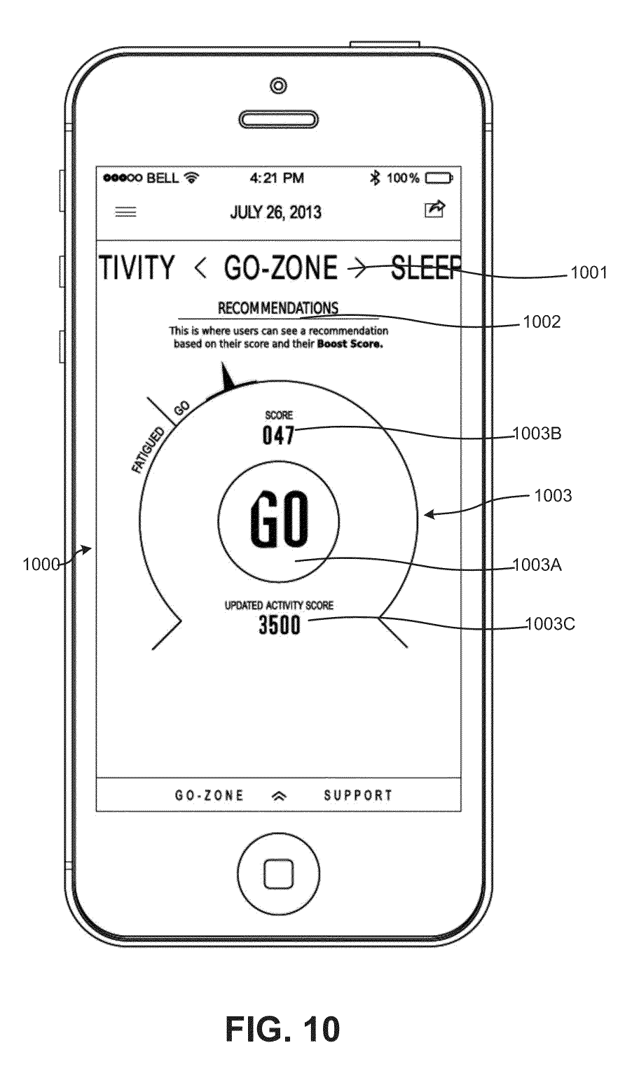

FIG. 10 illustrates an activity recommendation and fatigue level display 1000 that may be associated with an activity recommendation and fatigue level display module 213. In various embodiments, display 1000 may visually present to a user the user's current fatigue level and a recommendation of whether or not engage in activity. It is worth noting that one or more modules of activity tracking application 210 may compute and/or track fatigue level based on data received from earphones 100, and make an activity level recommendation. For example, HRV data tracked at regular intervals may be compared with other biometric or biological data to determine how fatigued the user is. Additionally, the HRV data may be compared to pre-loaded or learned fatigue level profiles, as well as a user's specified activity goals. Particular systems and methods for implementing this functionality are described in greater detail in U.S. patent application Ser. No. 14/140,414, filed Dec. 24, 2013, titled “System and Method for Providing an Intelligent Goal Recommendation for Activity Level”, and which is incorporated herein by reference in its entirety.

As illustrated, display 1000 may comprise a display navigation area 1001 (as described above), a textual activity recommendation 1002, and a center fatigue and activity recommendation display 1003. Textual activity recommendation 1002 may, for example, display a recommendation as to whether a user is too fatigued for activity, and thus must rest, or if the user should be active. Center display 1003 may display an indication to a user to be active (or rest) 1003A (e.g., “go”), an overall score 1003B indicating the body's overall readiness for activity, and an activity goal score 1003C indicating an activity goal for the day or other period. In various embodiments, indication 1003A may be displayed as a result of a binary decision—for example, telling the user to be active, or “go”—or on a scaled indicator—for example, a circular dial display showing that a user should be more or less active depending on where a virtual needle is pointing on the dial.

In various embodiments, display 1000 may be generated by measuring the user's HRV at the beginning of the day (e.g., within 30 minutes of waking up.) For example, the user's HRV may be automatically measured using the optical heartrate sensor 122 after the user wears the earphones in a position that generates a good signal as described in method 400. In embodiments, when the user's HRV is being measured, computing device 200 may display any one of the following: an instruction to remain relaxed while the variability in the user's heart signal (i.e., HRV) is being measured, an amount of time remaining until the HRV has been sufficiently measured, and an indication that the user's HRV is detected. After the user's HRV is measured by earphones 100 for a predetermined amount of time (e.g., two minutes), one or more processing modules 215 of computing device 200 may determine the user's fatigue level for the day and a recommended amount of activity for the day. Activity recommendation and fatigue level display 1000 is generated based on this determination.

In further embodiments, the user's HRV may be automatically measured at predetermined intervals throughout the day using optical heartrate sensor 122. In such embodiments, activity recommendation and fatigue level display 1000 may be updated based on the updated HRV received throughout the day. In this manner, the activity recommendations presented to the user may be adjusted throughout the day.

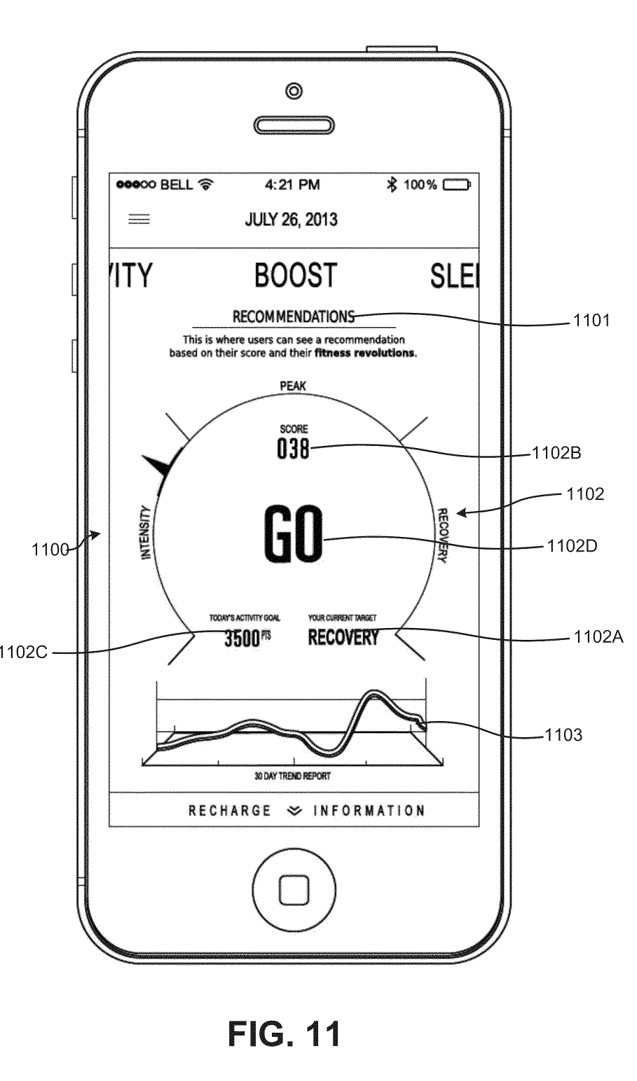

FIG. 11 illustrates a biological data and intensity recommendation display 800 that may be associated with a biological data and intensity recommendation display module 214. In various embodiments, display 800 may guide a user of the activity monitoring system through various fitness cycles of high-intensity activity followed by lower-intensity recovery based on the user's body fatigue and recovery level, thereby boosting the user's level of fitness and capacity on each cycle.

As illustrated, display 1100 may include a textual recommendation 1101, a center display 1102, and a historical plot 1103 indicating the user's transition between various fitness cycles. In various embodiments, textual recommendation 1101 may display a current recommended level of activity or training intensity based on current fatigue levels, current activity levels, user goals, pre-loaded profiles, activity scores, smart activity scores, historical trends, and other bio-metrics of interest. Center display 802 may display a fitness cycle target 1102A (e.g., intensity, peak, fatigue, or recovery), an overall score 1102B indicating the body's overall readiness for activity, an activity goal score 1102C indicating an activity goal for the day or other period, and an indication to a user to be active (or rest) 1102D (e.g., “go”). The data of center display 1102 may be displayed, for example, on a virtual dial, as text, or some combination thereof. In one particular embodiment implementing a dial display, recommended transitions between various fitness cycles (e.g., intensity and recovery) may be indicated by the dial transitioning between predetermined markers.

In various embodiments, display 1100 may display a historical plot 1103 that indicates the user's historical and current transitions between various fitness cycles over a predetermined period of time (e.g., 30 days). The fitness cycles, may include, for example, a fatigue cycle, a performance cycle, and a recovery cycle. Each of these cycles may be associated with a predetermined score range (e.g., overall score 1102B). For example, in one particular implementation a fatigue cycle may be associated with an overall score range of 0 to 33, a performance cycle may be associated with an overall score range of 34 to 66, and a recovery cycle may be associated with an overall score range of 67 to 100. The transitions between the fitness cycles may be demarcated by horizontal lines intersecting the historical plot 1103 at the overall score range boundaries. For example, the illustrated historical plot 1103 includes two horizontal lines intersecting the historical plot. In this example, measurements below the lowest horizontal line indicate a first fitness cycle (e.g., fatigue cycle), measurements between the two horizontal lines indicate a second fitness cycle (e.g., performance cycle), and measurements above the highest horizontal line indicate a third fitness cycle (e.g., recovery cycle).

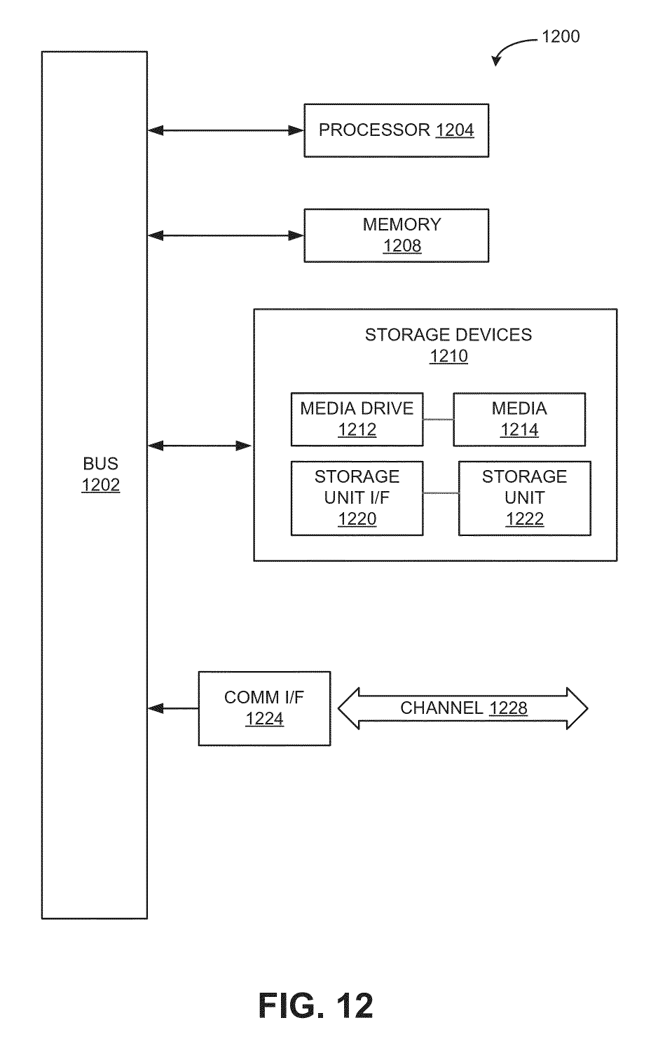

FIG. 12 illustrates an example computing module that may be used to implement various features of the systems and methods for estimating sky probes disclosed herein. As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 12. Various embodiments are described in terms of this example-computing module 1200. After reading this description, it will become apparent to a person skilled in the relevant art how to implement the application using other computing modules or architectures.

Referring now to FIG. 12, computing module 1200 may represent, for example, computing or processing capabilities found within desktop, laptop, notebook, and tablet computers; hand-held computing devices (tablets, PDA's, smart phones, cell phones, palmtops, etc.); mainframes, supercomputers, workstations or servers; or any other type of special-purpose or general-purpose computing devices as may be desirable or appropriate for a given application or environment. Computing module 1200 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 1200 might include, for example, one or more processors, controllers, control modules, or other processing devices, such as a processor 1204. Processor 1204 might be implemented using a general-purpose or special-purpose processing engine such as, for example, a microprocessor, controller, or other control logic. In the illustrated example, processor 1204 is connected to a bus 1202, although any communication medium can be used to facilitate interaction with other components of computing module 1200 or to communicate externally.

Computing module 1200 might also include one or more memory modules, simply referred to herein as main memory 1208. For example, preferably random access memory (RAM) or other dynamic memory, might be used for storing information and instructions to be executed by processor 1204. Main memory 1208 might also be used for storing temporary variables or other intermediate information during execution of instructions to be executed by processor 1208. Computing module 1200 might likewise include a read only memory (“ROM”) or other static storage device coupled to bus 852 for storing static information and instructions for processor 1208.

The computing module 1200 might also include one or more various forms of information storage mechanism 1210, which might include, for example, a media drive 1212 and a storage unit interface 1220. The media drive 1212 might include a drive or other mechanism to support fixed or removable storage media 1214. For example, a hard disk drive, a solid state drive, a magnetic tape drive, an optical disk drive, a CD, DVD, or Blu-ray drive (R or RW), or other removable or fixed media drive might be provided. Accordingly, storage media 1214 might include, for example, a hard disk, a solid state drive, magnetic tape, cartridge, optical disk, a CD, DVD, Blu-ray or other fixed or removable medium that is read by, written to or accessed by media drive 1212. As these examples illustrate, the storage media 1214 can include a computer usable storage medium having stored therein computer software or data.

In alternative embodiments, information storage mechanism 1210 might include other similar instrumentalities for allowing computer programs or other instructions or data to be loaded into computing module 1200. Such instrumentalities might include, for example, a fixed or removable storage unit 1222 and an interface 1220. Examples of such storage units 1222 and interfaces 1220 can include a program cartridge and cartridge interface, a removable memory (for example, a flash memory or other removable memory module) and memory slot, a PCMCIA slot and card, and other fixed or removable storage units 1222 and interfaces 1220 that allow software and data to be transferred from the storage unit 1222 to computing module 1200.

Computing module 1200 might also include a communications interface 1224. Communications interface 1224 might be used to allow software and data to be transferred between computing module 1200 and external devices. Examples of communications interface 1224 might include a modem or softmodem, a network interface (such as an Ethernet, network interface card, WiMedia, IEEE 602.XX or other interface), a communications port (such as for example, a USB port, IR port, RS232 port BLUETOOTH® interface, or other port), or other communications interface. Software and data transferred via communications interface 1224 might typically be carried on signals, which can be electronic, electromagnetic (which includes optical) or other signals capable of being exchanged by a given communications interface 1224. These signals might be provided to communications interface 1224 via a channel 1228. This channel 1228 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 1208, storage unit 1220, media 1214, and channel 1228. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 1200 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An system for providing a smart activity score, the system comprising:
a biometric sensor coupled to an earphone configured to be worn in an ear of a user;
a movement monitoring module that monitors a movement to determine a metabolic loading associated with the movement during a score period;
a period activity score module that creates and updates a period activity score based on the metabolic loading and the movement, the period activity score created and updated for the score period; and
a smart activity score module that creates and updates a smart activity score by aggregating a set of period activity scores.
wherein at least one of the movement monitoring module, the period activity score module, and the smart activity score module is embodied in the sensor.

2. The system claim 1, wherein the metabolic loading is determined from a set of metabolic loadings, each metabolic loading determined according to user information from a user.

3. The system of claim 2,
wherein the smart activity score is associated with a measuring period; and
wherein the smart activity score module calculates an average smart activity score from a set of past smart activity scores, each past smart activity score associated with a past measuring period.

4. The system of claim 3,
wherein the user information comprises a user lifestyle selected from a set of reference lifestyles; and
wherein the system for providing the smart activity score further comprises a user lifestyle module that:
associates each reference lifestyle with a lower threshold score and an upper threshold score, wherein the lower threshold score and the upper threshold score associated with each reference lifestyle define a range or scores, and wherein no two ranges of scores overlap; and
changes the user lifestyle to the reference lifestyle associated with the lower threshold score not greater than the average smart activity score and the upper threshold score not less than the average smart activity score.

5. The system of claim 1, further comprising:
a period activity score multiplier module that applies a period activity score multiplier to the period activity score to create an adjusted period activity score;
wherein the smart activity score comprises an aggregation of adjusted period activity scores.

6. The system of claim 5, wherein the period activity score multiplier is directly proportional to the number of continuous score periods over which a user activity type of the movement and a user activity intensity of the movement is maintained.

7. The system of claim 5, wherein the period activity score multiplier is directly proportional to smart activity score for the current measuring period.

8. The system of claim 1, wherein the score period is ten seconds.

9. A method for providing a smart activity score, the method comprising:
detecting movement based on data received from a biometric sensor coupled to an earphone configured to be worn in an ear of a user;
monitoring detected movement to determine a metabolic loading associated with the movement during a score period;
creating and updating a period activity score based on the metabolic loading and the movement, the period activity score created and updated for the score period; and
creating and updating a smart activity score by aggregating a set of period activity scores.

10. The method of claim 9,
wherein the metabolic loading is determined from a set of metabolic loadings, each metabolic loading determined according to user information from a user;
wherein the user information comprises a user lifestyle selected from a set of reference lifestyles; and
wherein the step of determining the set of metabolic loadings is based on the user lifestyle.

11. The method of claim 10, further comprising:
associating each reference lifestyle with a lower threshold score and an upper threshold score, wherein the lower threshold score and the upper threshold score associated with each reference lifestyle define a range of scores, and wherein no two ranges of scores overlap;
calculating an average smart activity score from a set of past smart activity scores, each past smart activity score associated with a past measuring period; and
changing the user lifestyle to the reference lifestyle associated with the lower threshold score not greater than the average smart activity score and the upper threshold score not less than the average smart activity score.

12. The method of claim 9, further comprising comparing the smart activity score to a past smart activity score, the smart activity score associated with a measuring period, the past smart activity score associated with a past measuring period.

13. The method of claim 9, further comprising:
receiving a second smart activity score from a second user; and
comparing the smart activity score to the second smart activity score.

14. The method of claim 9, wherein the step of creating and updating the smart activity score further comprises:
applying a score period multiplier to the score period to create an adjusted period activity score;
wherein the smart activity score comprises an aggregation of adjusted period activity scores.

15. The method of claim 14, wherein the score period multiplier is directly proportional to the number of continuous score periods over which a user activity type of the movement and a user activity intensity of the movement is maintained.

16. The method of claim 14, wherein the score period multiplier is directly proportional to the smart activity score for the current measuring period.

17. The method of claim 9, wherein the score period is ten seconds.

18. The method of claim 9, wherein at least one of the steps of monitoring the movement, creating and updating the period activity score, and creating and updating the smart activity score comprises using a biometric sensor embodied in an earphone.

19. A system for providing a smart activity score, the system comprising:
a processor; and
at least one computer program residing on the processor;
wherein the computer program is stored on a non-transitory computer readable medium having computer executable program code embodied thereon, the computer executable program code configured to:
monitor a movement to determine a metabolic loading associated with the movement during a score period;
create and update a period activity score based on the metabolic loading and the movement during the score period;
create and update a smart activity score by aggregating a set of period activity scores;

20. A system for providing a smart activity score, the system comprising:
an earphone comprising:
a biometric sensor;
a first processor;
a wireless transceiver;
a computing device configured to communicate wirelessly with the earphone, the computing comprising:
a display;
a memory;
a second processor;
wherein at least one computer program resides at least one of the first processor and second processor, wherein the computer program is stored on a non-transitory computer readable medium having computer executable program code embodied thereon, the computer executable program code configured to:
monitor a movement to determine a metabolic loading associated with the movement during a score period;
create and update a period activity score based on the metabolic loading and the movement during the score period; and
create and update a smart activity score by aggregating a set of period activity scores.

21. The system of claim 20, wherein the computing device is a mobile phone with a touch sensitive display.

22. The system of claim 20, wherein the metabolic loading is determined from a set of metabolic loadings, each metabolic loading determined according to user information from a user.

23. The system of claim 20, wherein the smart activity score is associated with a measuring period; and
wherein the computer executable program code is configured to calculate an average smart activity score from a set of past smart activity scores, each past smart activity score associated with a past measuring period.

24. The system of claim 20, wherein the user information comprises a user lifestyle selected from a set of reference lifestyles;

25. The system of claim 20, wherein the computer executable program code is further configured to:
associate each reference lifestyle with a lower threshold score and an upper threshold score, wherein the lower threshold score and the upper threshold score associated with each reference lifestyle define a range or scores, and wherein no two ranges of scores overlap; and
change the user lifestyle to the reference lifestyle associated with the lower threshold score not greater than the average smart activity score and the upper threshold score not less than the average smart activity score.

26. The system of claim 20, wherein the computer executable program code is further configured to:
apply a period activity score multiplier to the period activity score to create an adjusted period activity score;
wherein the smart activity score comprises an aggregation of adjusted period activity scores.

27. The system of claim 26, wherein the period activity score multiplier is directly proportional to the number of continuous score periods over which a user activity type of the movement and a user activity intensity of the movement is maintained.

28. The system of claim 26, wherein the period activity score multiplier is directly proportional to smart activity score for the current measuring period.

29. The system of claim 27, wherein the score periods are ten seconds.

30. The system of claim 20, wherein the computer executable program code is embodied on a computer readable medium embodied in a sensor configured to be attached to the body of a user.
